(19) United States
(12) Patent Application Publication
(10) Pub. No.: US 2025/0354165 A1
(43) Pub. Date: Nov. 20, 2025

(54) TRANSGENIC MOUSE MODELS SUPPORTING INNATE IMMUNE FUNCTION

(71) Applicant: The Jackson Laboratory, Bar Harbor, ME (US)

(72) Inventor: Leonard D. Shultz, Bar Harbor, ME (US)

(73) Assignee: The Jackson Laboratory, Bar Harbor, ME (US)

(21) Appl. No.: 19/236,089

(22) Filed: Jun. 12, 2025

Related U.S. Application Data
(63) Continuation of application No. 17/430,364, filed on Aug. 12, 2021, now Pat. No. 12,378,574.
(60) Provisional application No. 62/805,257, filed on Feb. 13, 2019.

(51) Int. Cl. C12N 15/85; A01K 67/0275; C12N 5/0783

(52) U.S. Cl. CPC C12N 15/8509; A01K 67/0275; C12N 5/0636; A01K 2217/05; A01K 2227/105; A01K 2267/03; C12N 2517/02

(57) ABSTRACT

Provided herein, in some aspects, is a NOD.Cg-Prkdc^{scid} Il2rg^{tm1Wj1}/SzJ (NOD scid gamma or NSG™) mouse comprising a nucleic acid encoding human FLT3L and an inactivated mouse Flt3 allele, methods of producing the mouse, and methods of using the mouse.

### RELATED APPLICATIONS

This application is a continuation of U.S. application Ser. No. 17/430,364, filed Aug. 12, 2021, which is a national stage filing under 35 U.S.C. § 371 of international application number PCT/US2020/018033, filed Feb. 13, 2020, which claims the benefit under 35 USC § 119 (e) of U.S. provisional application No. 62/805,257, filed Feb. 13, 2019, each of which is incorporated by reference herein in its entirety.

### GOVERNMENT LICENSE RIGHTS

This invention was made with government support under grant number 1R01132963 awarded by National Institutes of Health. The government has certain rights in the invention.

### REFERENCE TO AN ELECTRONIC SEQUENCE LISTING

The contents of the electronic sequence listing (J022770068US02-SEQ-HJD.xml; Size: 24,941 bytes; and Date of Creation: Jun. 10, 2025) is herein incorporated by reference in its entirety.

### BACKGROUND

Signaling through the fms-related tyrosine kinase 3 (FLT3) receptor supports survival, proliferation, and differentiation of hematopoietic progenitor cells and dendritic cells (DCs) (1, 2). Human DCs are necessary to present antigen to human T cells and are required for the development of a robust human immune response (4). Mature human DCs also produce interleukin 15 and other factors that support development of natural killer (NK) cells and other components of a human innate immune system (5).

### SUMMARY

Provided herein, in some embodiments, is an immunodeficient NOD.Cg-Prkdc^{scid} Il2rg^{tm1Wj1}/SzJ (NSG™) mouse (a “NOD scid gamma” mouse), comprising a nucleic acid encoding human FLT3L (e.g., comprising a human FLT3Z transgene) and an inactivated mouse Flt3 allele. Although the NSG™ mouse supports human hematopoietic stem cell (HSC) engraftment, it exhibits impaired development of human HSCs into dendritic cell (DC) populations (3). To provide a mouse model that supports development of human HSCs into DC populations, a transgenic NSG™ mouse expressing human FLT3L (NSG™-Tg(Hu-FLT3L)) was generated. Unexpectedly, however, engraftment of human HSCs was significantly lower in the NSG™-Tg(Hu-FLT3L) mouse compared with the NSG™ control. In an effort to understand the phenotype observed in the NSG™-Tg(Hu-FLT3L) mouse, the endogenous mouse receptor for FLT3L—Flt3—was knocked out. Surprisingly, engraftment with human HSCs of this transgenic line, referred to herein as NSG™ Flt3^{null}-Tg(Hu-FLT3L), results in (1) significantly increased percentages of both human CD3⁺ T cells and human CD33⁺ myeloid cells, (2) increased percentages of human CD123⁺ plasmacytoid dendritic cells, CD56⁺ human natural killer (NK) cells, CD14⁺ human monocyte macrophages, and CD11C⁺ HLA-DR⁺ human myeloid dendritic cells, and (3) support of mucosal engraftment of human CD45⁺ cells in the small intestines. Without being bound by theory, the decreased human HSC engraftment NSG™-Tg(Hu-FLT3L) may have been a consequence of human FLT3L activating, through the host mouse FLT3 receptor, the host mouse DCs and possibly other innate immune mouse components.

Thus, some aspects of the present disclosure provide a NSG™ mouse comprising a nucleic acid encoding human FLT3L and an inactivated mouse Flt3 allele (NSG™ Flt3^{null}-Tg(Hu-FLT3L)). This mouse model supports development of HSCs into many different cell types of the human innate immune system, including dendritic cells.

In some embodiments, the mouse comprises a genomic modification that inactivates the mouse Flt3 allele. The genomic modification, in some embodiments, is in at least one region of the mouse Flt3 allele selected from coding regions, non-coding regions, and regulatory regions. In some embodiments, the genomic modification is in at least one coding region of the mouse Flt3 allele. For example, the genomic modification may be in exon 6, exon 7, and/or exon 8. In some embodiments, the genomic modification is selected from genomic deletions, genomic insertions, genomic substitutions, and combinations thereof. For example, the genomic modification may be a genomic deletion. The mouse Flt3 allele may comprise, for example, a genomic deletion of nucleotide sequences in exon 6, exon 7, and exon 8.

In some embodiments, the nucleic acid sequence of SEQ ID NO: 5 has been deleted from the mouse Flt3 allele.

In some embodiments, the modified mouse Flt3 allele comprises the nucleic acid sequence of SEQ ID NO: 6.

In some embodiments, the nucleic acid encoding human FLT3L comprises a human FLT3L transgene. In some embodiments, the human FLT3L transgene comprises a nucleic acid sequence of SEQ ID NO: 7. In some embodiments, the mouse expresses human FLT3L. The human FLT3L may be expressed, for example at a level of at least 10,000 pg/ml. In some embodiments, the human FLT3L is expressed at a level of 10,000 pg/ml to 30,000 pg/ml. For example, the human FLT3L may be expressed at a level of 15,000+/−1000 pg/mL to 17,000+/−100 pg/ml.

In some embodiments, the mouse expresses mouse FLT3L. In some embodiments, the mouse FLT3L is expressed at a level of at least 2,000 pg/ml. For example, the mouse FLT3L may be expressed at a level of 5,000 pg/ml to 10,000 pg/ml. In some embodiments, the mouse FLT3L is expressed at a level of 6,000 pg/ml to 8,000 ml.

In some embodiments, the mouse does not express a detectable level of mouse FLT3. In some embodiments, a detectable level of mouse FLT3 expressed by the mouse is less than 1,000 pg/ml.

In some embodiments, the mouse lacks a detectable number of CD135⁺ multipotent progenitor cells.

In some embodiments, the mouse further comprises human CD34⁺ hematopoietic stem cells. The human CD34⁺ hematopoietic stem cells, in some embodiments, are from human umbilical cord blood, bone marrow, or mobilized peripheral blood.

In some embodiments, the mouse comprises a population of human CD45⁺ cells. The population of human CD45⁺ cells comprises, in some embodiments, human CD45⁺/CD3⁺ T cells and/or human CD45⁺/CD33⁺ myeloid cells.

In some embodiments, the population of human CD45⁺ cells comprises an increased percentage of human CD45⁺/CD3⁺ T cells, relative to a NOD scid gamma control mouse or a NOD scid gamma-Hu-FLT3L control mouse. For example, the percentage of human CD45⁺/CD3⁺ T cells in the mouse may be increased by at least 25%, at least 50%, or at least 100%.

In some embodiments, the population of human CD45⁺ cells comprises an increased percentage of human CD45⁺/CD33⁺ myeloid cells, relative to a NOD scid gamma control mouse or a NOD scid gamma-Hu-FLT3L control mouse. For example, the percentage of human CD45⁺/CD33⁺ myeloid cells in the mouse may be increased by at least 25%, at least 50%, or at least 100%.

In some embodiments, the mouse comprises an increased percentage of human CD123⁺ plasmacytoid dendritic cells, relative to a NOD scid gamma control mouse or a NOD scid gamma-Hu-FLT3L control mouse. For example, the percentage of human CD123⁺ plasmacytoid dendritic cells in the mouse may be increased by at least 25%, at least 50%, or at least 100%.

In some embodiments, the mouse comprises an increased percentage of human CD56⁺ natural killer cells, relative to a NOD scid gamma control mouse or a NOD scid gamma-Hu-FLT3L control mouse. For example, the percentage of human CD56⁺ natural killer cells in the mouse may be increased by at least 25%, at least 50%, or at least 100%.

In some embodiments, the mouse comprises an increased percentage of human CD14⁺ monocyte macrophages, relative to a NOD scid gamma control mouse or a NOD scid gamma-Hu-FLT3L control mouse. For example, the percentage of human CD14⁺ monocyte macrophages in the mouse may be increased by at least 25%, at least 50%, or at least 100%.

In some embodiments, the mouse comprises an increased percentage of human CD11C⁺ HLA-DR⁺ myeloid dendritic cells, relative to a NOD scid gamma control mouse or a NOD scid gamma-Hu-FLT3L control mouse. For example, the percentage of human CD11C⁺ HLA-DR⁺ myeloid dendritic cells in the mouse is increased by at least 25%, at least 50%, or at least 100%.

In some embodiments, the mouse exhibits mucosal engraftment of human CD45⁺ cells in the small intestines of the mouse.

Other aspects of the present disclosure provide a method comprising sublethally irradiating the mouse of any one of claims 1-23, and injecting the mouse with human CD34+ hematopoietic stem cells.

In some embodiments, the method further comprises administering to the mouse an agent of interest. In some embodiments, the method further comprises assessing an effect of the agent on human immune cells in the mouse.

In some embodiments, the method further comprises the human immune cells are selected from T cells, dendritic cells, natural killer cells, and macrophages.

Yet other aspects of the present disclosure provide a method comprising injecting a pronucleus of a NOD.Cg-Prkdc^{scid} Il2rg^{tm1Wj1}/SzJ (NOD scid gamma) mouse with a nucleic acid encoding human FLT3L, producing a NSG Tg(Hu-FLT3L) mouse, and inactivating a mouse Flt3 allele in the NSG Tg(Hu-FLT3L) mouse.

Further other aspects of the present disclosure provide a method comprising inactivating a mouse Flt3 allele in a NOD.Cg-Prkdc^{scid} Il2rg^{tm1Wj1}/SzJ (NOD scid gamma) mouse to produce a NSG Flt3^{null} mouse, and injecting a pronucleus of the NSG Flt3^{null} mouse with a nucleic acid encoding human FLT3L.

Still other aspects of the present disclosure provide a method comprising breeding female mice homozygous for Prkdc^{scid}, homozygous for Il2rg^{tm1Wj1}, homozygous for Flt3^{null}, and homozygous for a human FLT3L transgene with male mice homozygous for Prkdc^{scid}, hemizygous for the X-linked Il2rg^{tm1Wj1}, homozygous for Flt3^{null}, and homozygous for a human FLT3L transgene to produce progeny mice.

Further still, some aspects of the present disclosure provide a NOD.Cg-Prkdc^{scid} Il2rg^{tm1Wj1}/SzJ cell comprising a nucleic acid encoding human FLT3L and an inactivated endogenous Flt3 allele.

Yet other aspects of the present disclosure a transgenic rodent comprising a cell comprising a nucleic acid encoding human FLT3L and an inactivated endogenous Flt3 allele. In some embodiments, the transgenic rodent is a transgenic mouse.

Some aspects of the present disclosure provide a gRNA targeting mouse Flt3, optionally wherein the gRNA targets exon 6 or exon 8 or mouse Flt3. In some embodiments, the gRNA comprises the sequence of SEQ ID NO: 1. In some embodiments, the gRNA comprises the sequence of SEQ ID NO: 2.

Also provided herein is a mouse oocyte comprising any one of the gRNAs described herein. In some embodiments, the mouse oocyte is fertilized.

Some aspects further provide a mouse oocyte comprising a first gRNA targeting exon 6 of mouse Flt3 and a second gRNA targeting exon 8 of mouse Flt3, optionally wherein the mouse oocyte is fertilized.

A mouse oocyte, in some embodiments, further comprises Cas9 mRNA and/or Cas9 protein.

A mouse oocyte, in some embodiments, further comprises a human FLT3L transgene.

### BRIEF DESCRIPTION OF THE DRAWINGS

FIG. 1 shows human FLT3L levels in sera from NSG™-transgenic mice expressing human FLT3L (NSG™-Tg(Hu-FLT3L) that are hemizygous (labeled as Het) or homozygous (labeled as hom) for human FLT3L from founder lines 7 and 8. Each bar represents values from 3-4 female or male mice at 5 to 15 weeks of age.

FIG. 2 shows decreased levels of human hematopoietic stem cell (HSC) engraftment in homozygous NSG™-Tg(Hu-FLT3L) mice that are expressed as percentages of human CD45⁺ cells in blood. Each data point represents the percentage of human CD45⁺ cells in individual mice tested at 6-15 weeks post-engraftment. Both male and female mice were used.

FIG. 3 shows the absence of mouse CD135⁺ myeloid multipotential (MMP3) progenitor cells in NSG™ Flt3^{null}-Tg(Hu-FLT3L) mouse bone marrow. Each bar represents values from three (3) female mice at ten (10) weeks of age.

FIG. 4A shows the levels of mouse FLT3L in NSG™ Flt3^{null}-Tg(Hu-FLT3L) and NSG™ control mice. FIG. 4B shows the levels of human FLT3L in NSG™ Flt3^{null}-Tg(Hu-FLT3L) and NSG™ control mice.

FIG. 5A shows human CD45⁺ T-cell development in NSG™ Flt3^{null}-Tg(Hu-FLT3L) mice engrafted with human HSC and CD45⁺ T-cell development in NSG™ control mice. Eight (8) NSG™ Flt3^{null}-Tg(Hu-FLT3L) mice and ten (10) NSG™ control mice were injected at 8-12 weeks of age. Both male and female mice were used. FIG. 5B shows an increased percentage of human CD3⁺ T cell development in NSG™ Flt3^{null}-Tg(Hu-FLT3L) mice engrafted with human HSC compared with NSG™ control mice. FIG. 5C shows a decreased percentage of human CD20⁺ B-cell development in NSG™ Flt3^{null}-Tg(Hu-FLT3L) mice engrafted with human HSC compared with NSG™ control mice. This may be due to an increase in the percentage of HSCs differentiating into innate immune system cells (e.g., dendritic cells, natural killer cells, monocytes, macrophages, eosinophils, basophils, neutrophils) as opposed to adaptive immune system cells (e.g., T cells and B cells). FIG. 5D shows increased percentage of human CD33⁺ myeloid cell development in NSG™ Flt3^{null}-Tg(Hu-FLT3L) mice engrafted with human HSC compared with NSG™ control mice.

FIG. 6A shows increased human CD123⁺ plasmacytoid dendritic cell (DC) development in NSG™ Flt3^{null}-Tg(Hu-FLT3L) mice engrafted with human HSC compared with NSG™ control mice. Eight (8) NSG™ Flt3^{null}-Tg(Hu-FLT3L) mice and ten (10) NSG™ control mice were injected at 8-12 weeks of age. Both male and female mice were used. FIG. 6B shows increased human CD56⁺ natural killer cell (NK) development in NSG™ Flt3^{null}-Tg(Hu-FLT3L) mice engrafted with human HSC compared with NSG™ control mice. FIG. 6C shows increased human CD14⁺ monocyte/macrophage cell development in NSG™ Flt3^{null}-Tg(Hu-FLT3L) mice engrafted with human HSC compared with NSG™ control mice. FIG. 6D shows increased human CD11c/HLA-DR⁺ myeloid DC cell development in NSG™ Flt3^{null}-Tg(Hu-FLT3L) mice engrafted with human HSC compared with NSG™ control mice.

FIG. 7 shows the small intestine from a NSG™ Flt3^{null}-Tg(Hu-FLT3L) mouse and a NSG™ control mouse showing mucosal engraftment with HSC. Eight (8) NSG™ Flt3^{null}-Tg(Hu-FLT3L) mice and ten (10) NSG™ control mice were injected at 8-12 weeks of age. Both male and female mice were used.

FIG. 8A shows comparable human CD45⁺ cell development in NSG™ Flt3^{null}-Tg(Hu-FLT3L) mice engrafted with human HSC compared with NSG™ control mice. FIG. 8B shows increased human CD33⁺ myeloid cell development in NSG™ Flt3^{null}-Tg(Hu-FLT3L) mice engrafted with human HSC compared with NSG™ control mice. FIG. 8C shows increased human CD3⁺ T cell development in NSG™ Flt3^{null}-Tg(Hu-FLT3L) mice engrafted with human HSC compared with NSG™ control mice. FIG. 8D shows increased human CD14⁺ monocyte cell development in NSG™ Flt3^{null}-Tg(Hu-FLT3L) mice engrafted with human HSC compared with NSG™ control mice. FIG. 8E shows increased human CD11c+ HLA-DR⁺ myeloid dendritic cell development in NSG™ Flt3^{null}-Tg(Hu-FLT3L) mice engrafted with human HSC compared with NSG™ control mice. FIG. 8F shows increased human CD56⁺ natural killer (NK) cell development in NSG™ Flt3^{null}-Tg(Hu-FLT3L) mice engrafted with human HSC compared with NSG™ control mice.

### DETAILED DESCRIPTION

### NSG™ Flt3^{null}-Tg(Hu-FLT3L) Mouse

The present disclosure provides a NOD.Cg-Prkdc^{scid} Il2rg^{tm1Wj1}/SzJ (NSG™) mouse comprising a nucleic acid encoding human FLT3L and an inactivated mouse Flt3 allele. This mouse is referred to herein as a NSG™ Flt3^{null}-Tg(Hu-FLT3L) mouse.

The NSG™ mouse is an immunodeficient mouse that lack mature T cells, B cells, and natural killer (NK) cells, is deficient in multiple cytokine signaling pathways, and has many defects in innate immunity (see, e.g., Shultz L D et al. Nat. Rev. Immunol. 2007; 7 (2): 118-130; Shultz L D et al. 2005; J. Immunol. 174 (10): 6477-89; and Shultz L D et al. J. Immunol. 1995; 154 (1): 180-91). The NSG™ mouse, derived from the non-obese diabetic (NOD) mouse strain NOD/ShiLtJ (see, e.g., Makino S et al. Jikken Dobutsu 1980; 29 (1): 1-13), include the Prkdc^{scid} mutation (also referred to as the “severe combined immunodeficiency” mutation or the “scid” mutation) and the Il2rg^{tm1Wj1} targeted mutation. The Prkdc^{scid} mutation is a loss-of-function mutation in the mouse homolog of the human PRKDC gene—this mutation essentially eliminates adaptive immunity (see, e.g., Greiner D L et al. 1998; Stem Cells 16 (3): 166-177; and Blunt T et al. 1995; Cell 80 (5): 813-23). The Il2rg^{tm1Wj1} mutation is a null mutation in the gene encoding the interleukin 2 receptor gamma chain (IL2Rγ, homologous to IL2RG in humans), which blocks NK cell differentiation, thereby removing an obstacle that prevents the efficient engraftment of primary human cells (Shultz L D et al. 2005; Greiner et al. 1998; and Cao X. et al. Immunity 1995; 2 (3): 223-38). A loss-of-function mutation, as is known in the art, results in a gene product with little or no function. By comparison, a null mutation results in a gene product with no function. An inactivated allele may be a loss-of-function allele or a null allele.

The NSG™ Flt3^{null}-Tg(Hu-FLT3L) mouse provided herein comprises a nucleic acid encoding human FLT3L. FLT3L (e.g., NC_000019.10; chromosome:GRCh38:19:49473607: 49486831:1) is a cytokine and growth factor that stimulates the production of immune cells (e.g., B cells and T cells) by binding and activating the FLT3 receptor (see, e.g., Klein O. et al. Eur J Immunol. 2013; 43 (2): 533-539). FLT3L is important for the development of steady-state dendritic cells. In some embodiments, the nucleic acid encoding human FLT3L comprises a human FLT3L transgene. Surprisingly, the data described herein show that human FLT3L is capable of binding mouse FLT3, which, without being bound by theory, may activate innate mouse immunity. Thus, in some embodiments, the NSG™ mouse provided herein comprises a nucleic acid (e.g., DNA) encoding human FLT3L and an inactivated mouse Flt3 allele.

A nucleic acid may be DNA, RNA, or a chimera of DNA and RNA. In some embodiments, a nucleic acid (e.g., DNA) encoding human FLT3L comprises a gene encoding FLT3L. A gene is a sequence of nucleotides (DNA or RNA) that encodes a molecule (e.g., a protein) having a function. A gene may be endogenous (occurring naturally in a host organism) or exogenous (transferred, naturally or through genetic engineering, to a host organism). An allele is one of two or more alternative forms of a gene that arise by mutation and are found at the same locus on a chromosome. A gene, in some embodiments, includes a promoter sequence, coding regions (e.g., exons), non-coding regions (e.g., introns), and regulatory regions (also referred to as regulatory sequences). As is known in the art, a promoter sequence is a DNA sequence at which transcription of a gene begins. Promoter sequences are typically located directly upstream of (at the 5′ end of) a transcription initiation site. An exon is a region of a gene that codes for amino acids. An intron (and other non-coding DNA) is a region of a gene that does not code for amino acids.

A mouse comprising a human gene is considered to comprise a human transgene. A transgene is a gene exogenous to a host organism. That is, a transgene is a gene that has been transferred, naturally or through genetic engineering, to a host organism. A transgene does not occur naturally in the host organism (the organism, e.g., mouse, comprising the transgene). In some embodiments, a mouse as provided herein, comprises a FLT3L transgene, such as a human FLT3L transgene. In some embodiments, the human FLT3L transgene is integrated into the mouse genome. In some embodiments, the human FLT3L transgene comprises the nucleic acid sequence of SEQ ID NO: 7.

An inactivated allele is an allele that does not produce a detectable level of a functional gene product (e.g., a functional protein). In some embodiments, an inactivated allele is not transcribed. In some embodiments, an inactivated allele does not encode a functional protein. Thus, a mouse comprising an inactivated mouse Flt3 allele does not produce a detectable level of functional FLT3. In some embodiments, a mouse comprising an inactivated mouse Flt3 allele does not produce any functional FLT3.

In some embodiments, a mouse (e.g., a NSG™ Flt3^{null}-Tg(Hu-FLT3L) mouse) comprises a genomic modification that inactivates the mouse Flt3 allele. A modification, with respect to a nucleic acid, is any manipulation of the nucleic acid, relative to the corresponding wild-type nucleic acid (e.g., the naturally-occurring nucleic acid). A genomic modification is thus any manipulation of a nucleic acid in a genome, relative to the corresponding wild-type nucleic acid (e.g., the naturally-occurring nucleic acid) in the genome. Non-limiting examples of nucleic acid (e.g., genomic) modifications include deletions, insertions, “indels” (deletion and insertion), and substitutions (e.g., point mutations). In some embodiments, a deletion, insertion, indel, or other modification in a gene results in a frameshift mutation such that the gene no longer encodes a functional product (e.g. protein). Modifications also include chemical modifications, for example, chemical modifications of at least one nucleobase. Methods of nucleic acid modification, for example, those that result in gene inactivation, are known and include, without limitation, RNA interference, chemical modification, and gene editing (e.g., using recombinases or other programmable nuclease systems, e.g., CRISPR/Cas, TALENs, and/or ZFNs). In some embodiments, CRISPR/Cas gene editing is used to inactivate the mouse Flt3 allele, as described elsewhere herein.

In some embodiments, a genomic modification (e.g., a deletion or an indel) is in a (at least one) region of the mouse Flt3 allele selected from coding regions, non-coding regions, and regulatory regions. In some embodiments, the genomic modification (e.g., a deletion or an indel) is a coding region of the mouse Flt3 allele. For example, the genomic modification (e.g., a deletion or an indel) may be in exon 6, exon 7, exon 8, or it may span exons 6-8 of the mouse Flt3 allele. In some embodiments, the genomic modification is a genomic deletion. For example, the mouse Flt3 allele may comprise a genomic deletion of nucleotide sequences in exon 6, exon 7, and exon 8. In some embodiments, the nucleotide sequence of SEQ ID NO: 5 has been deleted from an inactivated mouse Flt3 allele. In some embodiments, an inactivated mouse Flt3 allele comprises the nucleotide sequence of SEQ ID NO: 6.

A NSG™ Flt3^{null}-Tg(Hu-FLT3L) mouse provided herein, in some embodiments, expresses human FLT3L. In some embodiments, human FLT3L is expressed at a level of at least 5,000 pg/ml or at least 10,000 pg/ml. For example, human FLT3L may be expressed at a level of at least 5,000 pg/ml, 7,500 pg/ml, 10,000 pg/ml, 12,500 pg/ml, 15,000 pg/ml, 17,500 pg/ml, 20,000 pg/ml, 22,500 pg/ml, 25,000 pg/ml, 27,500 pg/ml, 30,000 pg/ml, 32,500 pg/ml, 35,000 pg/ml, 37,500 pg/ml, 40,000 pg/ml, 42,500 pg/ml, 45,000 pg/ml, 47,500 pg/ml, or 50,000 pg/ml. In some embodiments, human FLT3L is expressed at a level of 10,000 pg/ml to 30,000 pg/ml. In some embodiments, human FLT3L is expressed at a level of 15,000+/−1000 pg/mL to 17,000+/−100 pg/ml. Methods of detecting FLT3L protein expression are known and may be used as provided herein. For example, flow cytometry and/or an ELISA (enzyme-linked immunosorbent assay) using an anti-FLT3L antibody may be used to detect the level of human FLTL3 protein present in mouse tissue and/or blood.

In some embodiments, a NSG™ Flt3^{null}-Tg(Hu-FLT3L) mouse may also expression mouse FLTL3. In some embodiments, mouse FLT3L is expressed at a level of at least 1,000 pg/ml or at least 2,000 pg/ml. For example, mouse FLT3L may be expressed at a level of 3,000 pg/ml, 4,000 pg/ml, 5,000 pg/ml, 6,000 pg/ml, 7,000 pg/ml, 8,000 pg/ml, 9,000 pg/ml, or 10,000 pg/ml. In some embodiments, mouse FLT3L is expressed at a level of 5,000 pg/ml to 10,000 pg/ml. In some embodiments, mouse FLT3L is expressed at a level of 6,000 pg/ml to 8,000 ml.

In some embodiments, a NSG™ Flt3^{null}-Tg(Hu-FLT3L) mouse does not express a detectable level of mouse FLT3. A detectable level of mouse FLT3 is any level of FLT3 protein detected using a standard protein detection assay, such as flow cytometry and/or an ELISA. In some embodiments, a NSG™ Flt3^{null}-Tg(Hu-FLT3L) mouse expresses an undetectable level or a low level of mouse FLT3. For example, a mouse may express less than 1,000 pg/ml mouse FLT3. In some embodiments, a NSG™ Flt3^{null}-Tg(Hu-FLT3L) mouse expresses less than 500 pg/ml mouse FLT3 or less than 100 pg/ml mouse FLT3. The mouse FLT3 receptor is also referred to as cluster of differentiation antigen CD135. Thus, in some embodiments, a NSG™ Flt3^{null}-Tg(Hu-FLT3L) mouse does not comprise (there is an absence of) CD135⁺ multipotent progenitor (MPP3) cells.

### Human Innate Immune System Model

The NSG™ Flt3^{null}Tg(Hu-FLT3L) mouse of the present disclosure, in some embodiments, is used to support engraftment of human CD34⁺ HSCs and development of a human innate immune system. The human immune system includes the innate immune system and the adaptive immune system. The innate immune system is responsible for recruiting immune cells to sites of infection, activation of the complement cascade, the identification and removal of foreign substances from the body by leukocytes, activation of the adaptive immune system, and acting as a physical and chemical barrier to infectious agents.

In some embodiments, the NSG™ Flt3^{null}Tg(Hu-FLT3L) mouse is sublethally irradiated (e.g., 100-300 cGy) to kill resident mouse HSCs, and then the irradiated mouse is engrafted with human CD34⁺ HSCs (e.g., 50,000 to 200,000 HSCs) to initiate the development of a human innate immune system. Thus, in some embodiments, the mouse further comprises human CD34⁺ HSCs. Human CD34⁺ HSCs may be from any source including, but not limited to, human umbilical cord blood, mobilized peripheral blood, and bone marrow. In some embodiments, the human CD34⁺ HSCs are from human umbilical cord blood.

The differentiation of human CD34⁺ HSCs into divergent immune cells (e.g., T cells, B cells, dendritic cells) is a complex process in which successive developmental steps are regulated by multiple cytokines. This process can be monitored through cell surface antigens, such as cluster of differentiation (CD) antigens. CD45, for example, is expressed on the surface of HSCs, macrophages, monocytes, T cells, B cells, natural killer cells, and dendritic cells, thus can be used as a marker indicative of engraftment. On T cells, CD45 regulates T cell receptor signaling, cell growth, and cell differentiation. In some embodiments, the NSG™ Flt3^{null}Tg(Hu-FLT3L) mouse comprises human CD45⁺ cells. Unexpectedly, the NSG™ Flt3^{null}Tg(Hu-FLT3L) mouse exhibits mucosal engraftment of human CD45⁺ cells in the small intestines. In some embodiments, the NSG™ Flt3^{null}Tg(Hu-FLT3L) mouse also exhibits engraftment of human CD45⁺ cells to tissues in the lung, thymus, spleen, and/or lymph nodes.

As CD45+ cells mature, they begin to express additional biomarkers, indicative of the various developmental stages and differentiating cell types. Developing T cells, for example, also express CD3, CD4, and CD8. As another example, developing myeloid cells express CD33⁺. The NSG™ Flt3^{null}Tg(Hu-FLT3L) mouse of the present disclosure, advantageously, comprises not only human CD45⁺ cells but also double positive human CD45⁺/CD3⁺ T cells as well as double positive human CD45⁺/CD33⁺ myeloid cells.

Thus, in some embodiments, a population of human CD45⁺ cells in a NSG™ Flt3^{null}Tg(Hu-FLT3L) mouse comprises human CD45⁺/CD3⁺ T cells. In some embodiments, the population of human CD45⁺ cells comprises an increased percentage of human CD45⁺/CD3⁺ T cells, relative to a NSG™ control mouse. In some embodiments, the percentage of human CD45⁺/CD3⁺ T cells in the NSG™ Flt3^{null}Tg(Hu-FLT3L) mouse is increased by at least 25%, relative to a NSG™ control mouse. For example, the percentage of human CD45⁺/CD3⁺ T cells in the NSG™ Flt3^{null}Tg(Hu-FLT3L) mouse may be increased by at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%, relative to a NSG™ control mouse. In some embodiments, the percentage of human CD45⁺/CD3⁺ T cells in the NSG™ Flt3^{null}Tg(Hu-FLT3L) mouse is increased by at least 50%, relative to a NSG™ control mouse. In some embodiments, the percentage of human CD45⁺/CD3⁺ T cells in the NSG™ Flt3^{null}Tg(Hu-FLT3L) mouse is increased by at least 100%, relative to a NSG™ control mouse. In some embodiments, the percentage of human CD45⁺/CD3⁺ T cells in the NSG™ Flt3^{null}Tg(Hu-FLT3L) mouse is increased by 25%-100%, 25%-75%, 25%-50%, 50%-100%, 50%-75%, or 75%-100%, relative to a NSG™ control mouse.

In some embodiments, a population of human CD45⁺ cells in a NSG™ Flt3^{null}Tg(Hu-FLT3L) mouse comprises human CD45⁺/CD33⁺ T cells. In some embodiments, the population of human CD45⁺ cells comprises an increased percentage of human CD45⁺/CD33⁺ T cells, relative to a NSG™ control mouse. In some embodiments, the percentage of human CD45⁺/CD33⁺ T cells in the NSG™ Flt3^{null}Tg(Hu-FLT3L) mouse is increased by at least 25%, relative to a NSG™ control mouse. For example, the percentage of human CD45⁺/CD33⁺ T cells in the NSG™ Flt3^{null}Tg(Hu-FLT3L) mouse may be increased by at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%, relative to a NSG™ control mouse. In some embodiments, the percentage of human CD45⁺/CD33⁺ T cells in the NSG™ Flt3^{null}Tg(Hu-FLT3L) mouse is increased by at least 50%, relative to a NSG™ control mouse. In some embodiments, the percentage of human CD45⁺/CD33⁺ T cells in the NSG™ Flt3^{null}Tg(Hu-FLT3L) mouse is increased by at least 100%, relative to a NSG™ control mouse. In some embodiments, the percentage of human CD45⁺/CD33⁺ T cells in the NSG™ Flt3^{null}Tg(Hu-FLT3L) mouse is increased by 25%-100%, 25%-75%, 25%-50%, 50%-100%, 50%-75%, or 75%-100%, relative to a NSG™ control mouse.

The NSG™ Flt3^{null}Tg(Hu-FLT3L) mouse provided herein, surprisingly, is also capable of supporting engraftment of dendritic cells (e.g., plasmacytoid dendritic cells and myeloid dendritic cells), natural killer cells, and monocyte-derived macrophages (monocyte macrophages). Plasmacytoid dendritic cells (pDCs) secrete high levels of interferon alpha; myeloid dendritic cells (mDCs) secrete interleukin 12, interleukin 6, tumor necrosis factor, and chemokines; natural killer cells destroy damaged host cells, such as tumor cells and virus-infected cells; and macrophages consume substantial numbers of bacteria or other cells or microbes.

In some embodiments, a NSG™ Flt3^{null}Tg(Hu-FLT3L) mouse comprises an increased percentage of human CD123⁺ plasmacytoid dendritic cells, relative to a NSG™ control mouse. In some embodiments, the percentage of human CD123⁺ plasmacytoid dendritic cells in the NSG™ Flt3^{null}Tg(Hu-FLT3L) mouse is increased by at least 25%, relative to a NSG™ control mouse. For example, the percentage of human CD123⁺ plasmacytoid dendritic cells in the NSG™ Flt3^{null}Tg(Hu-FLT3L) mouse may be increased by at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%, relative to a NSG™ control mouse. In some embodiments, the percentage of human CD123⁺ plasmacytoid dendritic cells in the NSG™ Flt3^{null}Tg(Hu-FLT3L) mouse is increased by at least 50%, relative to a NSG™ control mouse. In some embodiments, the percentage of human CD123⁺ plasmacytoid dendritic cells in the NSG™ Flt3^{null}Tg(Hu-FLT3L) mouse is increased by at least 100%, relative to a NSG™ control mouse. In some embodiments, the percentage of human CD123⁺ plasmacytoid dendritic cells in the NSG™ Flt3^{null}Tg(Hu-FLT3L) mouse is increased by 25%-100%, 25%-75%, 25%-50%, 50%-100%, 50%-75%, or 75%-100%, relative to a NSG™ control mouse.

In some embodiments, a NSG™ Flt3^{null}Tg(Hu-FLT3L) mouse comprises an increased percentage of human CD56+ natural killer cells, relative to a NSG™ control mouse. In some embodiments, the percentage of human CD56+ natural killer cells in the NSG™ Flt3^{null}Tg(Hu-FLT3L) mouse is increased by at least 25%, relative to a NSG™ control mouse. For example, the percentage of human CD56+ natural killer cells in the NSG™ Flt3^{null}Tg(Hu-FLT3L) mouse may be increased by at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%, relative to a NSG™ control mouse. In some embodiments, the percentage of human CD56+ natural killer cells in the NSG™ Flt3^{null}Tg(Hu-FLT3L) mouse is increased by at least 50%, relative to a NSG™ control mouse. In some embodiments, the percentage of human CD56+ natural killer cells in the NSG™ Flt3^{null}Tg(Hu-FLT3L) mouse is increased by at least 100%, relative to a NSG™ control mouse. In some embodiments, the percentage of human CD56+ natural killer cells in the NSG™ Flt3^{null}Tg(Hu-FLT3L) mouse is increased by 25%-100%, 25%-75%, 25%-50%, 50%-100%, 50%-75%, or 75%-100%, relative to a NSG™ control mouse.

In some embodiments, a NSG™ Flt3^{null}Tg(Hu-FLT3L) mouse comprises an increased percentage of human CD14⁺ monocyte macrophages, relative to a NSG™ control mouse. In some embodiments, the percentage of human CD14⁺ monocyte macrophages in the NSG™ Flt3^{null}Tg(Hu-FLT3L) mouse is increased by at least 25%, relative to a NSG™ control mouse. For example, the percentage of human CD14⁺ monocyte macrophages in the NSG™ Flt3^{null}Tg(Hu-FLT3L) mouse may be increased by at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%, relative to a NSG™ control mouse. In some embodiments, the percentage of human CD14⁺ monocyte macrophages in the NSG™ Flt3^{null}Tg(Hu-FLT3L) mouse is increased by at least 50%, relative to a NSG™ control mouse. In some embodiments, the percentage of human CD14⁺ monocyte macrophages in the NSG™ Flt3^{null}Tg(Hu-FLT3L) mouse is increased by at least 100%, relative to a NSG™ control mouse. In some embodiments, the percentage of human CD14⁺ monocyte macrophages in the NSG™ Flt3^{null}Tg(Hu-FLT3L) mouse is increased by 25%-100%, 25%-75%, 25%-50%, 50%-100%, 50%-75%, or 75%-100%, relative to a NSG™ control mouse.

In some embodiments, a NSG™ Flt3^{null}Tg(Hu-FLT3L) mouse comprises an increased percentage of human CD11C⁺ HLA-DR⁺ myeloid dendritic cells, relative to a NSG™ control mouse. In some embodiments, the percentage of human CD11C⁺ HLA-DR⁺ myeloid dendritic cells in the NSG™ Flt3^{null}Tg(Hu-FLT3L) mouse is increased by at least 25%, relative to a NSG™ control mouse. For example, the percentage of human CD11C⁺ HLA-DR⁺ myeloid dendritic cells in the NSG™ Flt3^{null}Tg(Hu-FLT3L) mouse may be increased by at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%, relative to a NSG™ control mouse. In some embodiments, the percentage of human CD11C⁺ HLA-DR⁺ myeloid dendritic cells in the NSG™ Flt3^{null}Tg(Hu-FLT3L) mouse is increased by at least 50%, relative to a NSG™ control mouse. In some embodiments, the percentage of human CD11C⁺ HLA-DR⁺ myeloid dendritic cells in the NSG™ Flt3^{null}Tg(Hu-FLT3L) mouse is increased by at least 100%, relative to a NSG™ control mouse. In some embodiments, the percentage of human CD11C⁺ HLA-DR⁺ myeloid dendritic cells in the NSG™ Flt3^{null}Tg(Hu-FLT3L) mouse is increased by 25%-100%, 25%-75%, 25%-50%, 50%-100%, 50%-75%, or 75%-100%, relative to a NSG™ control mouse.

### Methods of Producing Transgenic Animals

Provided herein, in some aspects, are methods of producing a transgenic animal that expresses human FLT3L. A transgenic animal, herein, refers to an animal that has a foreign (exogenous) nucleic acid (e.g. transgene) inserted into (integrated into) its genome. In some embodiments, the transgenic animal is a transgenic rodent, such as a mouse or a rat. In some embodiments, the transgenic animal is a mouse. Methods of producing transgenic mice, for example, are well known. Three conventional methods used for the production of transgenic animals include DNA microinjection (Gordon and Ruddle, Science 1981: 214: 1244-124, incorporated herein by reference), embryonic stem cell-mediated gene transfer (Gossler et al., Proc. Natl. Acad. Sci. 1986; 83: 9065-9069, incorporated herein by reference) and retrovirus-mediated gene transfer (Jaenisch, Proc. Natl. Acad. Sci. 1976; 73: 1260-1264, incorporated herein by reference), any of which may be used as provided herein.

The nucleic acid encoding human FLT3L, in some embodiments, comprises a human FLT3L transgene that comprise a promoter (e.g., a constitutively active promoter) operably linked to a nucleotide sequence encoding human FLT3L. In some embodiments, the nucleic acid encoding human FLT3L used to produce a transgenic animal (e.g., mouse) is present on an vector, such as a plasmid, a bacterial artificial chromosome (BAC), or a yeast artificial chromosome (YAC), which is delivered, for example, to the pronucleus/nucleus of a fertilized embryo where the nucleic acid randomly integrates into the animal genome. In some embodiments, the nucleic acid (e.g., carried on a BAC) is delivered to a fertilized embryo of a NSG™ mouse to produce a NSG™ Tg(Hu-FLT3L) mouse. Following injection of the fertilized embryo, the fertilized embryo is transferred to a pseudopregnant female, which subsequently gives birth to offspring comprising the nucleic acid encoding human FLTL3. The presence or absence of the nucleic acid encoding human FLTL3 may be confirmed, for example, using any number of genotyping methods (e.g., sequencing and/or genomic PCR).

Also provided herein are methods of inactivating an endogenous Flt3 allele. In some embodiments, an endogenous Flt3 allele is inactivated in a transgenic animal. In some embodiments, the transgenic animal is a NSG™ Tg(Hu-FLT3L) mouse. Thus, in some embodiments, the method comprise inactivating a mouse Flt3 allele in a NSG™ Tg(Hu-FLT3L) mouse to produce a NSG™ Flt3^{null}Tg(Hu-FLT3L) mouse. Methods of gene (allele) inactivation are known, any of which may be used as provided herein. In some embodiments, a gene/genome editing method is used. Engineered nuclease-based gene editing systems that may be used as provided herein include, for example, clustered regularly interspaced short palindromic repeat (CRISPR) systems, zinc-finger nucleases (ZFNs), and transcription activator-like effector nucleases (TALENs). See, e.g., Carroll D Genetics. 2011; 188 (4): 773-782; Joung J K et al. Nat Rev Mol Cell Biol. 2013; 14 (1): 49-55; and Gaj T et al. Trends Biotechnol. 2013 July; 31 (7): 397-405, each of which is incorporated by reference herein.

In some embodiments, a CRISPR system is used to inactivate an endogenous Flt3 allele, for example, to produce a NSG™ Flt3^{null}Tg(Hu-FLT3L) mouse. See, e.g., Harms D W et al., Curr Protoc Hum Genet. 2014; 83: 15.7.1-15.7.27; and Inui M et al., Sci Rep. 2014; 4: 5396, each of which are incorporated by reference herein). For example, Cas9 mRNA or protein and one or multiple guide RNAs (gRNAs) can be injected directly into mouse embryos to generate precise genomic edits into a Flt3 gene. Mice that develop from these embryos are genotyped or sequenced to determine if they carry the desired mutation(s), and those that do are bred to confirm germline transmission.

The CRISPR/Cas system is a naturally occurring defense mechanism in prokaryotes that has been repurposed as a RNA-guided-DNA-targeting platform for gene editing. Engineered CRISPR systems contain two main components: a guide RNA (gRNA) and a CRISPR-associated endonuclease (e.g., Cas protein). The gRNA is a short synthetic RNA composed of a scaffold sequence for nuclease-binding and a user-defined nucleotide spacer (e.g., ˜15-25 nucleotides, or ˜20 nucleotides) that defines the genomic target to be modified. Thus, one can change the genomic target of the Cas protein by simply changing the target sequence present in the gRNA. In some embodiments, the CRISPR-associated endonuclease is selected from Cas9, Cpf1, C2c1, and C2c3. In some embodiments, the Cas nuclease is Cas9.

A guide RNA comprises at least a spacer sequence that hybridizes to (binds to) a target nucleic acid sequence (e.g., a region of the Flt3 allele, such as the promoter sequence, a coding sequence, or a noncoding sequence), and a CRISPR repeat sequence that binds the endonuclease and guides the endonuclease to the target nucleic acid sequence. As is understood by the person of ordinary skill in the art, each gRNA is designed to include a spacer sequence complementary to its genomic target sequence (e.g., a region of the Flt3 allele). See, e.g., Jinek et al., Science, 2012; 337: 816-821 and Deltcheva et al. Nature, 2100; 471: 602-607, each of which is incorporated by reference herein. In some embodiments, a gRNA used in the methods provided herein binds to exon 6 of a mouse Flt3 allele. In some embodiments, a gRNA used in the methods provided herein binds to exon 7 of a mouse Flt3 allele. In some embodiments, a gRNA used in the methods provided herein binds to exon 8 of a mouse Flt3 allele. In some embodiments, multiple gRNAs are used to target multiple regions of the Flt3 allele. In some embodiments, two gRNAs are used, one binding to exon 6 and one binding to exon 8 of the Flt3 allele. In some embodiments, a gRNA of the present disclosure comprises the nucleotide sequence of SEQ ID NO: 1. In some embodiments, a gRNA of the present disclosure comprises the nucleotide sequence of SEQ ID NO: 2.

### Methods of Use

The mouse model provided herein may be used for any number of applications. For example, the NSG™ Flt3^{null}-Tg(Hu-FLT3L) mouse may be used to test how a particular agent (e.g., therapeutic agent) or medical procedure (e.g., tissue transplantation) affects the human innate immune system (e.g., human innate immune cell responses).

In some embodiments, the NSG™ Flt3^{null}-Tg(Hu-FLT3L) mouse is used to evaluate an effect of an agent on human innate immune system development. Thus, provided herein are methods that comprise administering an agent to the NSG™ Flt3^{null}-Tg(Hu-FLT3L) mouse, and evaluating an effect of the agent on human innate immune system development in the mouse. Effects of an agent may be evaluated, for example, by measuring a human innate immune cell (e.g., T cell and/or dendritic cell) response (e.g., cell death, cell signaling, cell proliferation, etc.). Non-limiting examples of agents include therapeutic agents, such as anti-cancer agents and anti-inflammatory agents, and prophylactic agents, such as immunogenic compositions (e.g., vaccines).

In other embodiments, the NSG™ Flt3^{null}-Tg(Hu-FLT3L) mouse is used to evaluate an immunotherapeutic response to a human tumor. Thus, provided herein are methods that comprise administering an agent to a NSG™ Flt3^{null}-Tg(Hu-FLT3L) mouse that has a human tumor, and evaluating an effect of the agent on the human innate immune system and/or on the tumor in the mouse. Effects of an agent may be evaluated by measuring a human innate immune cell (e.g., T cell and/or dendritic cell) response and/or tumor cell response (e.g., cell death, cell signaling, cell proliferation, etc.). In some embodiments, the agent is an anti-cancer agent.

In yet other embodiments, the NSG™ Flt3^{null}-Tg(Hu-FLT3L) mouse is used to evaluate a human innate immune response to an infectious microorganism. Thus, provided herein are methods that comprise exposing the NSG™ Flt3^{null}-Tg(Hu-FLT3L) mouse to an infectious microorganism (e.g., bacteria and/or virus), and evaluating an effect of the infectious microorganism on the human innate immune response. Effects of an infectious microorganism may be evaluated by measuring a human innate immune cell (e.g., T cell and/or dendritic cell) response (e.g., cell death, cell signaling, cell proliferation, etc.). These methods may further comprise administering a drug or an anti-microbial agent (e.g., an anti-bacterial agent or an anti-viral agent) to the mouse, and evaluating an effect of the drug or anti-microbial agent on the infectious microorganism.

In still further embodiments, the NSG™ Flt3^{null}-Tg(Hu-FLT3L) mouse is used to evaluate a human immune response to tissue transplantation. Thus, provided herein are methods that comprise transplanting tissue (e.g., allogeneic tissue) to a NSG™ Flt3^{null}-Tg(Hu-FLT3L) mouse, and evaluating an effect of the transplanted tissue on the human innate immune response. Effects of a transplanted tissue may be evaluated by measuring a human innate immune cell (e.g., T cell and/or dendritic cell) response (e.g., cell death, cell signaling, cell proliferation, etc.) to the transplanted tissue.

### EXAMPLES

### Example 1: Production of NSG™-Tg(Hu-FLT3L) Mice

To support human DC development and innate immune function, a panel of transgenic NSG™ mouse lines expressing human FLT3 ligand (FLT3L) was produced. To produce these transgenic mouse lines, a 13.8 kilobase (kb) BamHI restriction fragment from the bacterial artificial chromosome (BAC) clone RP11-360G9 obtained from CHORI BACPAK was subcloned into pBluescript to eliminate other genes in the BAC. Purified, linearized DNA was then injected into the pronuclei of NOD.Cg-Prkdc^{scid} Il2rg^{tm1Wj1}/SzJ (NSG™) fertilized eggs.

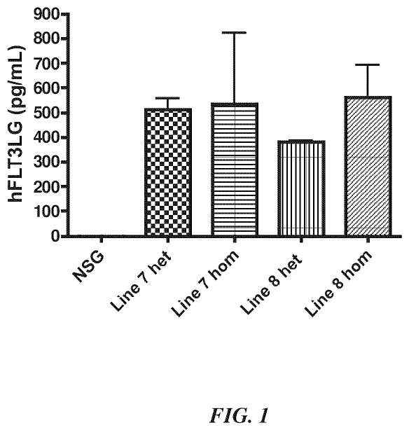

Eleven founders were identified by PCR. Of these founders, four founder lines (lines 3, 7, 8, and 10) had detectable levels of circulating human FLT3L when tested by enzyme linked immunosorbent assay (ELISA). These founders were mated with NSG non-transgenic mice. The transgenic offspring of this cross were tested by ELISA for circulating human FLT3L levels. The levels of FLT3L in transgenic NSG mice that were hemizygous or homozygous for the human FLT3L transgene (lines 7 and 8) ranged from 400 to 600 pg/mL (FIG. 1).

### Example 2: NSG™-Tg(Hu-FLT3L) Mice Exhibit Impaired Development of Human HSCs

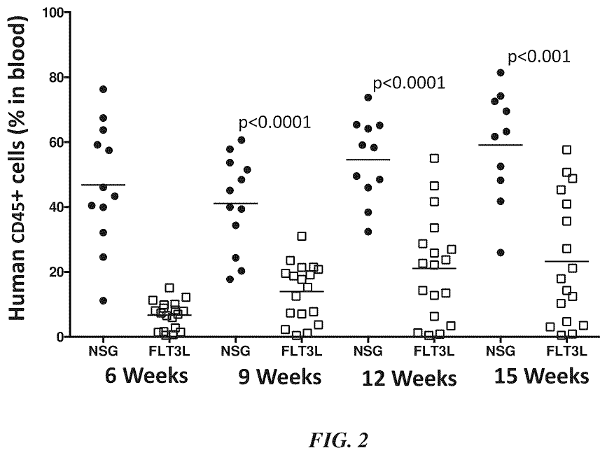

Transgenic NSG™ mice expressing human FLT3L (NSG™-Tg(Hu-FLT3L)) were then engrafted with human cord blood CD34⁺ HSCs. Cohorts of NSG™-Tg(Hu-FLT3L) mice and NSG™ non-transgenic control mice were sublethally irradiated (200cGy) and injected intravenously with 100,000 human umbilical cord blood HSCs. Flow cytometry of peripheral blood from engrafted mice was conducted at 6, 9, 12, and 15 weeks post-engraftment. Unexpectedly, the percentage of human CD45⁺ cells was significantly lower in the NSG™-Tg(Hu-FLT3L) mice compared with NSG™ controls at all time points tested (FIG. 2).

### Example 3: Production of NSG™ Flt3^{null}Tg(Hu-FLT3L) Mice

To test whether the decreased human HSC engraftment in NSG™-Tg(Hu-FLT3L) mice is a consequence of human FLT3L activating the host mouse DCs and possibly other innate immune mouse components, the gene encoding the mouse FLT3 receptor (Flt3) was knocked out (to prevent human FLT3L from binding to the mouse FLT3 receptor and activating mouse innate immunity). Without being bound by theory, without the mouse FLT3 receptor, both the human FLT3L and mouse FLT3L would be available to bind to the human FLT3 receptor.

The mouse Flt3 gene encoding the FLT3 receptor was knocked out directly in the NSG™-Tg(Hu-FLT3L) homozygous strain that expressed the human FLT3 ligand. To knock-out the mouse Flt3 gene, two single guide RNA (sgRNAs, see Table 1 below) were designed to target exons 6 and 8, which should eliminate the immunoglobulin-like domain, even if the transcript is retained and a hypomorph results. The sgRNAs were designed using breaking Cas (6) and Zifit (7) software to minimize the likelihood of off-target cutting and were also checked against the human sequence to ensure the human FLT3L transgene would not be targeted. Both sgRNAs were designed as Tru-Guides (“truncated”), each with only 19 base recognition sites, in order to further reduce the likelihood of off-target cutting (8). The sgRNAs were prepared as described previously (9).

The screening for mouse Flt3 knock-outs was performed using PCR primers (see Table 1 below) that flanked the region of interest (e.g., exons 6-8). The PCR primers yield a 1942 base pair (bp) wild-type amplicon, with a predicted size of 670 bp for the mutant dropout (“DO”) allele. Following PCR, samples were screened by Sanger sequencing using the same primers. Three founders carrying knock-out alleles were identified by PCR and subsequently sequenced. Three knock-out lines were established (Lines 1, 2, and 4) by intercrossing N2 mice from the founder with the largest deletion. Knock-outs were typed by PCR, and ultimately, a single knock-out line was expanded, validated, and characterized (NPD.Cg-Flt3^{em1Mvw} Prkdc^{scid}Il2rg^{tm1Wj1}Tg(Hu-FLT3L) 7Sz, abbreviated as NSG™ Flt3^{null}Tg(Hu-FLT3L) mice.

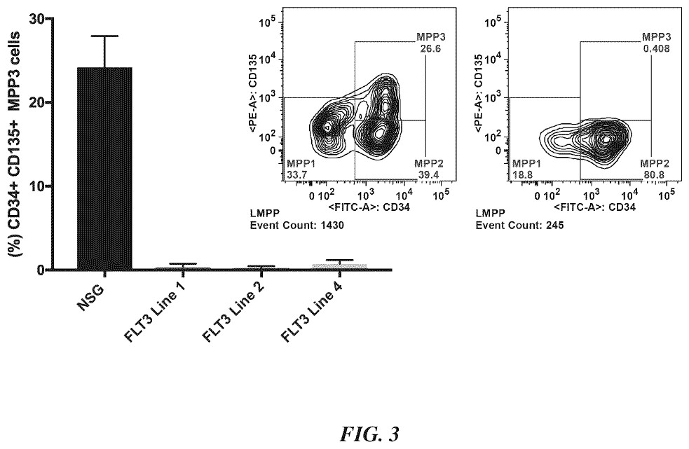

The modified Flt3^{null} allele lost 1500 bp beginning before exon 6 and termination just before the end of exon 8. The deleted gene sequence is shown as SEQ ID NO: 5 (1500 base pairs deleted in the mouse Flt3 receptor gene in NSG™ Flt3^{null}-Tg(Hu-FLT3L) mice). The resulting PCR product for the modified Flt3^{null} allele is 442 bp. The sequence of this PCR product is shown as SEQ ID NO: 6 (PCR product resulting from targeted mouse Flt3 gene deletion). Mouse FLT3 receptor is called cluster of differentiation antigen CD135. The NSG™ Flt3^{null} Tg(Hu-FLT3L) mice were validated for lack of mouse FLT3 by flow cytometry of bone marrow dendritic cells by absence of CD135+ multipotent progenitor (MPP3) cells (FIG. 3).

TABLE 1
Sequences used to generate and screen
Flt3 knock-out mice

| | | SEQ |
|---|---|---|
| Sequence | Name | ID NO: |
| 5′-GAACAGCUUGGUGCAUUCG | sgRNA-Exon 6 | 1 |
| 5′-GAUGGUCACCAACGCUGAC | sgRNA-Exon 8 | 2 |
| 5′-CCAACCTGAACTGTATGGAGATAG | PCR-Forward | 3 |
| 5′-CCACAGGGAAAGCCACTAAA | PCR-Reverse | 4 |

### Example 4: NSG™ Flt3^{null}Tg(Hu-FLT3L) Mice Support Dendritic Cell Development

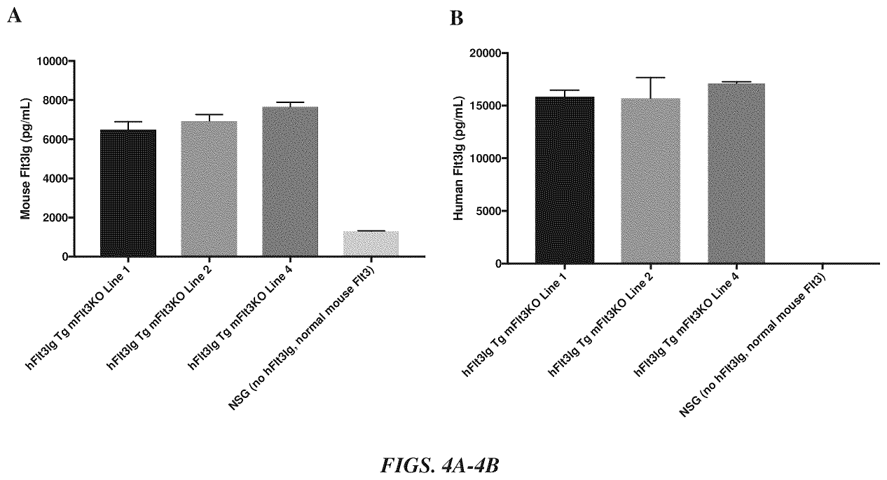

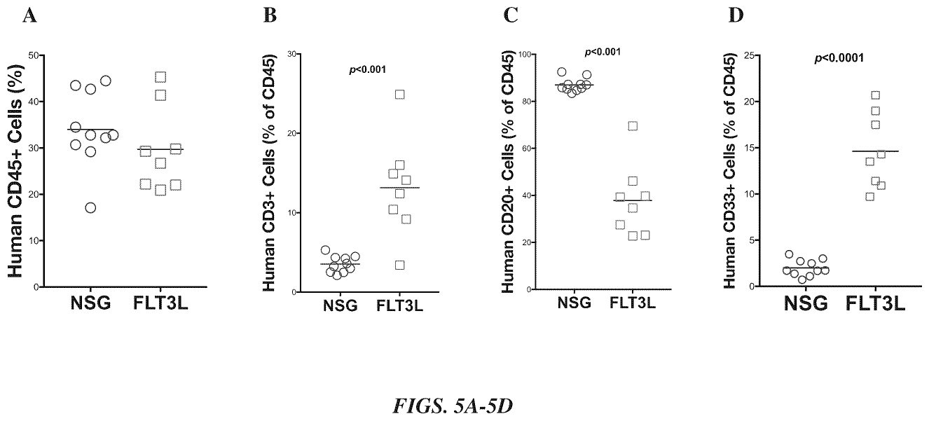

ELISA assays of the sera of the NSG™ Flt3^{null} Tg(Hu-FLT3L) mice expressing the human FLT3L transgene and lacking the mouse FLT3 receptor showed levels of the human FLT3L ranging from 15,175+/−1,137 pg/mL to 17,120+/−92.7 pg/mL. Sera from the NSG™ non-transgenic control mice showed no detectable FLT3L levels (FIGS. 4A-4B). The levels of mouse FLT3L ranged from 6,000 to 8,000 pg/mL in NSG™ Flt3^{null}Tg(Hu-FLT3L), while the level of mouse FLT3L in NSG™ control was ˜ 1,000 pg/mL.

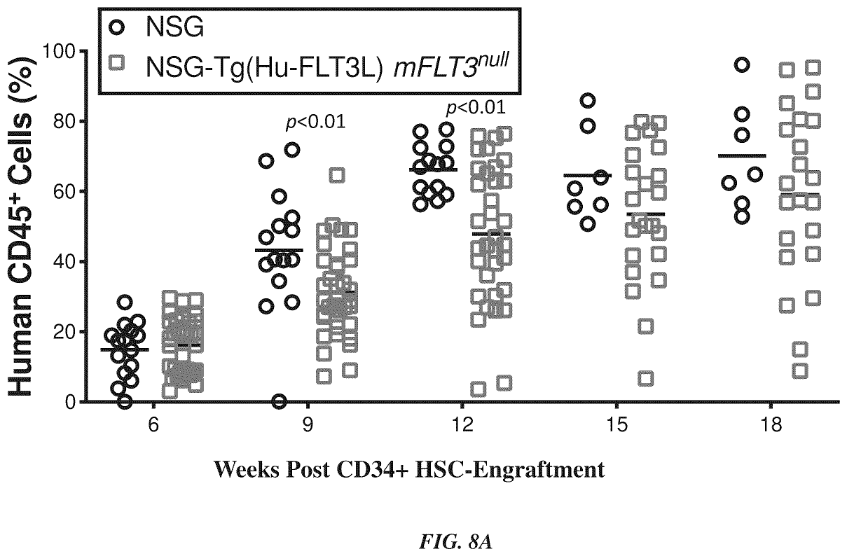

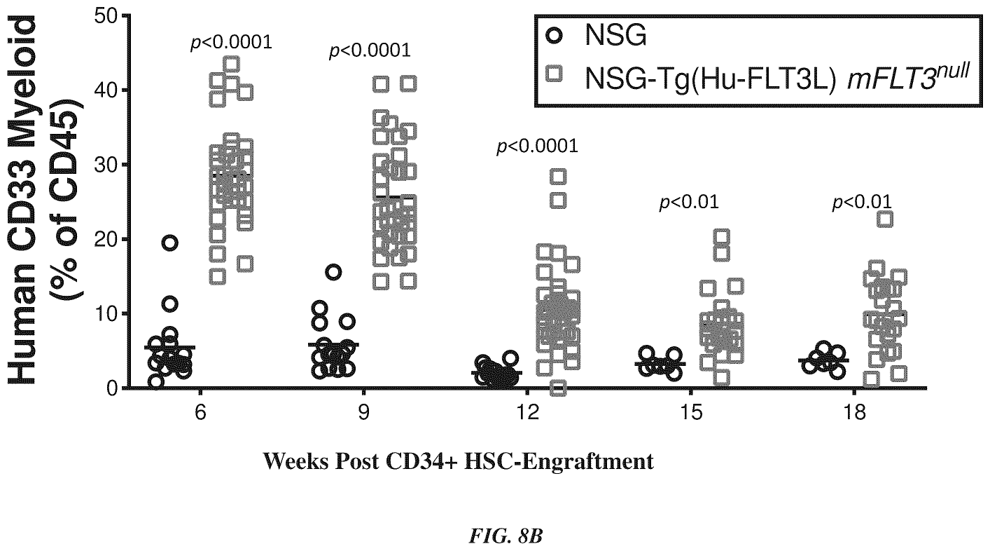

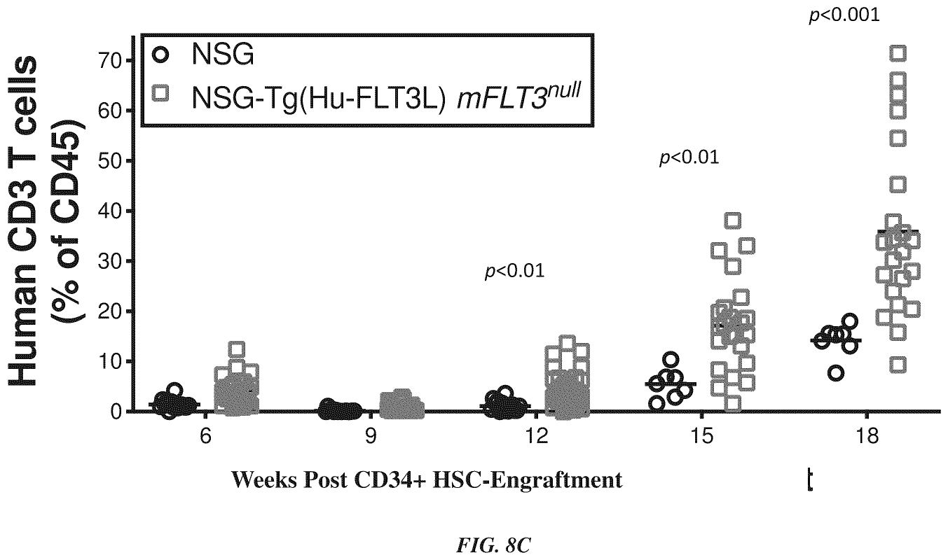

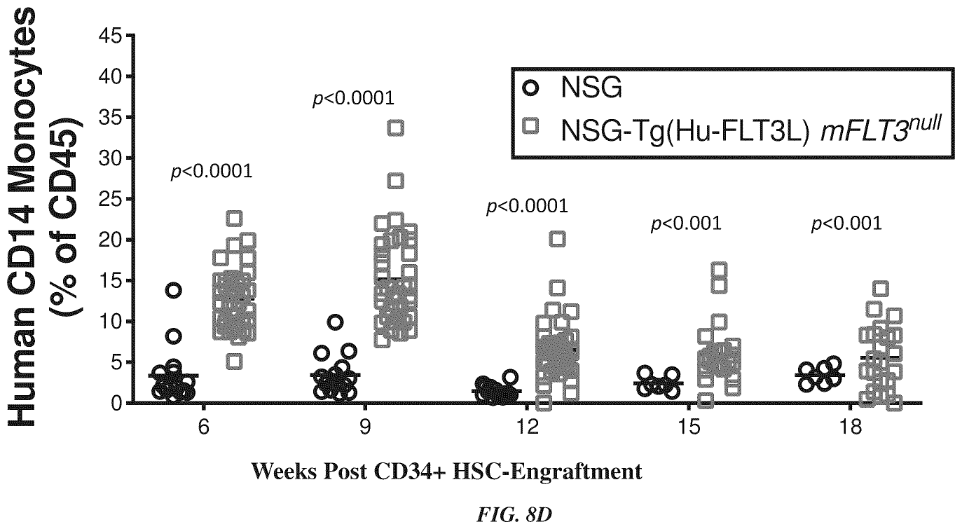

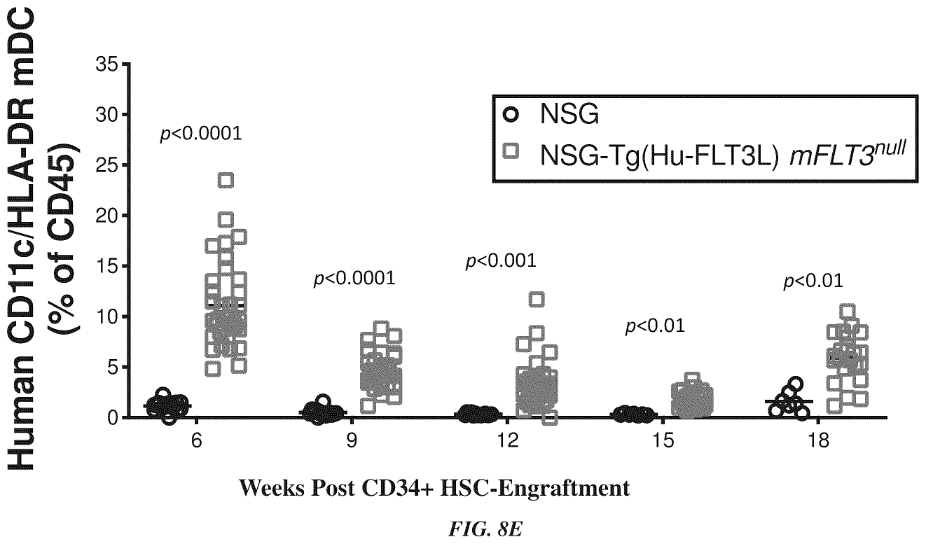

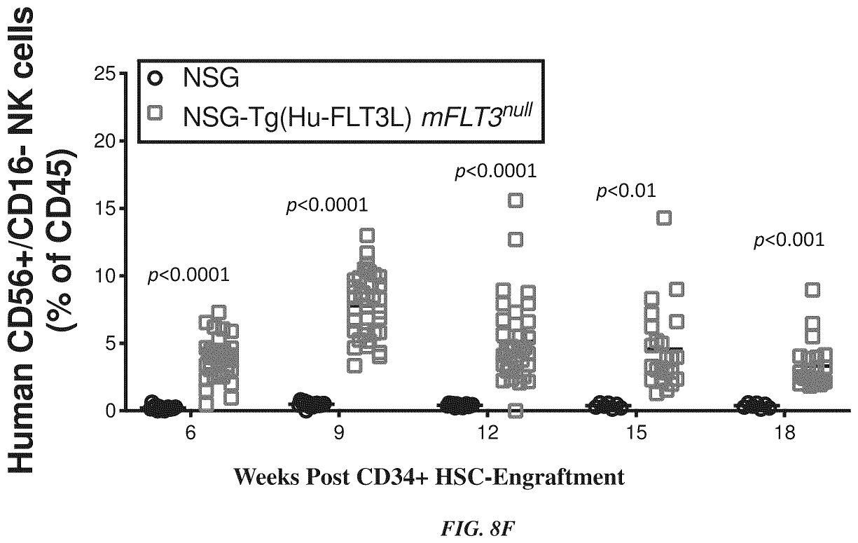

To determine the ability of the NSG™ Flt3^{null} Tg(Hu-FLT3L) mice to support engraftment with human CD34+ HSC and development of a human immune system, groups of NSG™ Flt3^{null} Tg(Hu-FLT3L) mice (n=8) and NSG™ control mice (n=10) at 8-12 weeks of age were sublethally irradiated (200cGy) and injected intravenously (IV) with 100,000 human umbilical cord blood CD34+ HSC. Flow cytometry analyses of the peripheral blood of the engrafted mice at 6, 9, 12, 15, and 18 weeks post-engraftment showed that the NSG™ Flt3^{null} Tg(Hu-FLT3L) and NSG™ control mice had similar percentages of human CD45+ leukocytes. However, in the human CD45+ cell population, the NSG™ Flt3^{null} Tg(Hu-FLT3L) mice had significantly increased percentages of both human CD3+ T cells and human CD33+ myeloid cells (FIGS. 5A-5D and FIGS. 8A-8C).

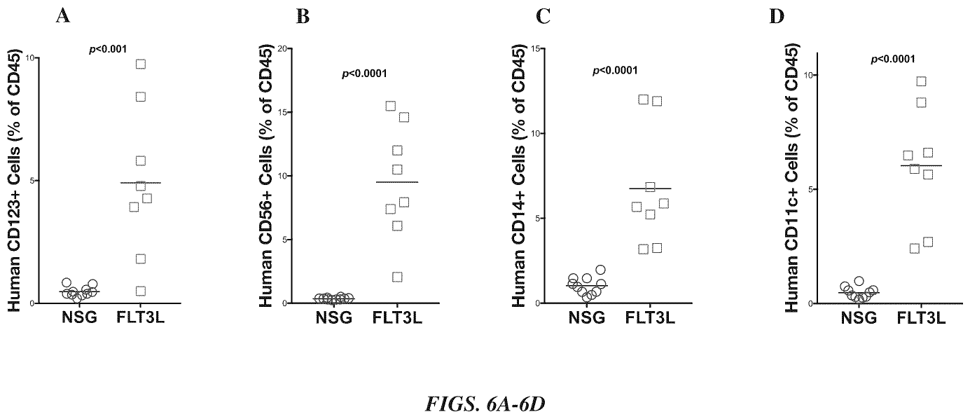

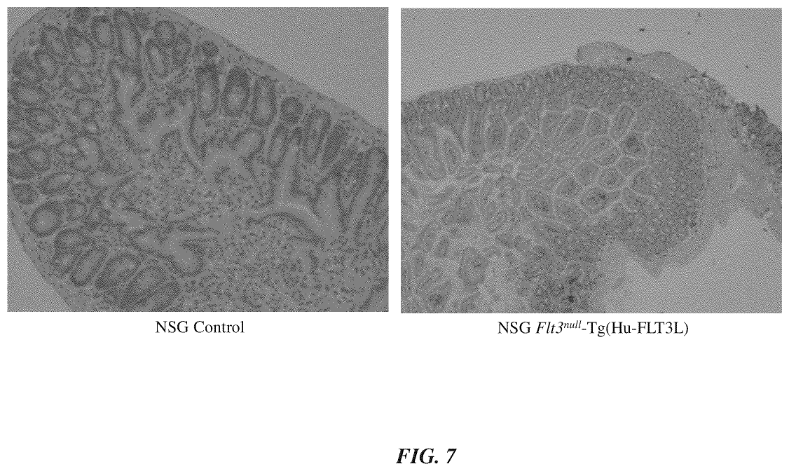

NSG™ Flt3^{null} Tg(Hu-FLT3L) mice also showed increased percentages of human CD123+ plasmacytoid dendritic cells, CD56+ human natural killer (NK) cells, CD14+ human monocyte macrophages, and CD11C+ HLA-DR+ human myeloid dendritic cells (FIGS. 6A-6D, 8D-8F). Moreover, histochemical analyses of tissues from the engrafted mice unexpectedly showed mucosal engraftment of human CD45+ cells in the small intestines of NSG™ Flt3^{null} Tg(Hu-FLT3L) mice (FIG. 7).

SEQUENCES

ATCCCCCAGTGCTGGTGACCTTGAACACAGGGTCTGCCCTGAGCAGTGCTGACAGCCTGTTAGCTGTCATTTTGAAG
CATTTCCTGAAGGAAACTTACCTTATCATTGCAGCTGTAAAGAAGAAGGCCCTGCTGTTGTCAGAAAGGAGGAAAAG
GTACTTCATGAGTTGTTCGGAACAGACATCAGATGCTGTGCTAGAAATGCACTGGGCCGCGAATGCACCAAGCTGTT
CACCATAGGTAATGGGGGACTGTCGGCTATGTGTCCTTAGTGACGGTTCCTAAGGGACCGGTGATAGCTTCTGTGTG
GGTAATCATTCCACTTCAGAAAGATGATGGAAAGTTATACTGAAATCCGAATCTAGATCGAATAGTCCACATTCTGA
CATAAAAGCAATTGAACACACAGGTATGGCCAAGCTTGGTGGCCCGGGCTTTTAATCCTTCTTTCATAGGACTTTGA
TTTGATTCAATTTGATTTGATTTGATTTGACTTAGGTGCTGGGCTCAAACCCAGGGACTCAGAGCATCTGCTTTGCC
ACTGAGTTCCACTCCAGATCTTTTGCACCACTTTATTTATTTATTTATTGTGTGTGTGTATACACACATTAAATACA
CACACATACACACACACACACACACACACACACACACACACACACACACACACACACGCTACAGCATGTAATGTGGA
TGTCAGGTTCTCTTCTTGTCATATGGGGCCCCTGGATCAAATTCAGGTCAGTCTGGCAGCGGCAGGCACCTCCACCC
ACTGAGCCATCTTGATGCCCCGTTCATACCATTTACAATGTAACACTGTAGATTCTGTAAGGGAGGATTTCTGCAAC
AAAGGGTATTCCATCTCGAGTGGGTGGTTTTACTCTGTCGCTTTTTTTTTTTTAGTTTTGTGCTGCTCTGTGACTCC
AGGTCTTCTCCAGGCCAGCCTCTTTTACCCTGATCTTCTCAGCTCTGCATTGTTTGGCTTGATTAGTGATGGCAGGG
ATGCTGACACCATTGCTTTTCAAAACGTTTGCGACACTTCTGAGCTGAGGCAAAAGTTTTTAAAAAAAAGATGTGCT
TTTTTTTTTTTTTTTTTGTCTTCTTTGGGATATTTTTATCTTCTAGATCTAAACCAGGCTCCTCAGAGCACACTGCC
CCAGTTATTCCTGAAAGTGGGGGAACCCTTGTGGATCAGGTGTAAGGCCATCCATGTGAACCATGGATTCGGGCTCA
CCTGGGAGCTGGAAGACAAAGCCCTGGAGGAGGTAACAGCGCCCACCCGTGGTGTTTACCGTAAGCTCTCAGCGTGG
GCTGGAAACCCGGAATACAGACTCCCCTTTCATTAATACTCAGGGCAGCTACTTTGAGATGAGTACCTACTCCACAA
ACAGGACCATGATTCGGATTCTCTTGGCCTTTGTGTCTTCCGTGGGAAGGAACGACACCGGATATTACACCTGCTCT
TCCTCAAAGCACCCCAGCCAGTCAGCGTTGGTGACCA (SEQ ID NO: 5)
CCAACCTGAACTGTATGGAGATAGCTTGTCTATGGGGAAAAAAAATCACCAAATAAAATAAAATCTAAAATATTAAA
TAATAAAGTAATACTGTAACATTATCCTAATTAGATACAGAGTAGCTAGCTGGTCCCTGCAGATTCCAGGCTGGATA
CAGTTGAGTTAATTTTTCATCTGCTCTTTTCTTCCATCCCTAAAAATATACTTTGTAACTTCATCCCCTAAACATAC
TGGGTCCCCCCACCCCATCCTAGGTAACGGGCTCACGAAATTTGGGGTTGGTTGGTTTGTTTGTTTTTAATGCTGTC
CTGGAACTCGCTGTGTAGATTGGTTGGCCTTGAACTCACAGAAATCTACCAGCATCTGCCTCCCCAGTGCTGGGATT
AAAGGCACAGGCCACCAAGCTTGACTCTGCTTGTGTCTTTAGTGGCTTTCCCTGTGG (SEQ ID NO: 6)
GATCCCTGTGCTCCTCCGAGTGGCACAGGAAACTTGGCTGATAGAGGCGCTCTCTCCATGCATCCTCCACCATCCAC
TCCCCGGTCCCTTAGACTTCCATTCAGAAGTGACACCTGTCACCTCCACCCTCATGTGACTGGCCAAAGTGAGTGTG
GCCATGTCCGAGTTCCCCAGGATGGGACCTCGAATCCTCCTGCTGGGGGGGTGGGGGCAGCAGATGTTTCAGGACAG
TCTGCCACGGATCAGATGGGTGAGCAAGAGATTCAGGATGGGCGTGATGGGTCACACCTGTAATCCCAGCATTTTGG
GAGGCCGAGGCAGGCTGATCACCTGAGGTCAGGAGTTCGAGATCAGCCTGGCCAACATAGTGAAACCTCGTCTCTAC
TAAAAATACAAAAATTAGCCGGGCATGGTGGCACGCACCTGCAATTCCAGCTACTTGGGAGCCTGAGGCACAAGAAT
TGCTTAAGCCCAGGAGGCGGAGGTTGCCGTGAGCCGAGACTGTGCCACTGCACTCCAGCCTGGGCAACAGAGTGAGA
CCCCATCTAAAAAAAAAAAATGCTTCAGAGATTCAGAGATCCACAGATGTGGAAGAGAGCTTCTATTTTGTCTCATG
TACCACCTCACCCCTCAGTTTGGAGTTGGAGAAGCCGAGGCTTAGAGAGGTCAAGGGGCTGGCCAAGCTCCCACAGT
GATAAAGCTAGTGCTAGGGCGACACCTGCCAGGCCCAATGTGACAAGATGCATCCTGAGTCCTGTGTGCCAAGCCTT
GGCGGGGCAAAGTTGGGGCCCGGAGGACGGTAAGACACAGCAACTGCGAGTTCCCAGAGACAGATCCGGGTTCCAGC
CCCAGCTCACCATGCACTGGGCGTGCCACCCAGGGAGTCAGCCCCACTGCAGCCCCCGCTGCGGGGCGTCCGGAGAA
CGCGCCATCTGCGGCGTGAGCGGCCGCCTCTCACCACCAGGGGGCGCGCTCCGCCTGGGCCCAGATTCCACCCCTTG
ACTGTCTCCCCCCAAAAATTTCCTTTCACTTTCGGTCTCTGGCTGTCACCCGGCTTGGCCCCTTCCACACCCAACTG
GGGCAAGCCTGGTGCGTGAGGAGACTTGGACTCTAGGTCCCCAAGGGGCGGAGCCAGGGTCCCCACTCCTGGGGCAG
GGGGAGCAGAGGGTGGGGGGCTGGGTTCCCGGGTCCCTAACCTGGGGAGGGTTCTGGGGACCAGACGTCGAGGTTCT
TAGAAGTGGAGATGAAGAGTTTTCACCGTAACTGGGGCAGACGCAGGAAGCCTGGGGGAGGAAGGGGCGGAAACTCA
GCCACATCAATGGGACGCGGGAGGGGCGGGGAGGCAAAGGGGCGGGCAGGGCAGGGGCTGGGGCATGAGGGTCCGAG
ACTTGTTCTTCTGTCCCTTCCAAGACCCGGCGACAGGAGGCATGAGGGGCCCCCGGCCGAAATGACAGTGCTGGCGC
CAGCCTGGAGCCCAACAGTGCGTAAACCCCAGGGACAAGATCAGGGGAGAGGGGAGGCACAATGTCAGGATGGGGCA
GAGATGAGGGGAGATGGACGGGAGAACAGATGGACAGATGACGAGGAAATAGGAGGGGAGATGGACAGATGTGAGGG
GAGATGGATAGGAGAGGAGACGGACAGAGGAGGGGGAGATGGACAGAGGATGGGGAGATGGACAGAGGAGGGGGAGA
TAGAGGAGAGGGAGATAGAGAGGAGTGGGAGATGGACAGGAGCGGGGAGATGGACAGAGGAGGGGGAGATGGACAGA
GGAGAGGGCAGATGAACAGAGGAGAGGGAGATAAAGAGGAGGGAGGTGGACAGGAGGAGGGAGATGGACAGAGAAGG
GGGAGATGGACAGAGGTGGGGGAGATGGACAGGAGGGAAGATGGAGAGGAGGGGGTATGGACAGAGGAGAGGGGAGA
TGGACAGGAGGGGGAGATGGACAGAGGAGAGGGAGATGAAGAGGAGGGGGAGGTGGACAGGAGGAGGGAGACGGACA
GAGGAGAGGAAGAGAGACAGAGGAGGGGGGAGATGGACAGAGGAGGGGGAGATGGACAGAAAAGGGGGGAGATGGAC
AGAGGAGAGGGAAGGTGGACAGAGCCAAGAACAAATGAAGAGGACGTGGACCAAGATCAAGAGAGAAGCAGGCAACA
GTGGTGTAGAAGGCAGAGGGAGGGACAGAGCTGGAGGAACCCGGGCGAGGAAACCAGACGTGAAGATGAGGTGGTTG
GAGAGAGACCAGCAGAGTGGGGGAGATGGGGAGAGAGAGGGGTGGGGCAGAGGGGGATGCAAACTGGACAGCATTGG
ACCAGAGGCAGAGAGAAACCGGGAAAGACAGGCAGAGATGGGGCCATGTCTCCAGAAAGTGTGGAAGAGGCAGAAGG
ACACCCAGGGAAGGAGGAGCGGGGAAGACAGAACAGTACAGGTGGGAAGCCCGGAGGAGGGGGCTGTGTGTGGAACA
GCAGAGGGCTCCCCCAGCACCCGCTCCCCTGCAGACCTATCTCCTCCTGCTGCTGCTGCTGAGCTCGGGACTCAGTG
GGACCCAGGACTGCTCCTTCCAACACAGCCCCATCTCCTCCGACTTCGCTGTCAAAATCCGTGAGCTGGTGAGCGGC
GCTGCCCCGGACCCCCTCATGTGATCCCCCTTCCCCCCACTTTTTTTTTTAAGTAGAGATGGGGTCTCTCTCCCTGT
GTTTCCCAGGGTGGTCTTGAACTCCTGGGCTCAAGCGATCCTCCCACCTTGGCCTCCCAAAGTGCTGGAATTACAGG
CGTGAGCCACTGTGCTTAGGGGTGTCATCCCTTTTTAAGGGCAAGGTTCTGTGGCTTCTTCTGGGCTCCCCCTCTCT
TGGTCTTGTCCCTCTCTCTCTGGATCTCTGCTGCCACCTCTGGGTCCCCACAGTTCTGTTTCTCGCTGTTTTCAGCC
AGGCCTGATCCTGTTTTCTCCCGCAGTCTGACTACCTGCTTCAAGATTACCCAGTCACCGTGGCCTCCAACCTGCAG
GACGTAAGTCATGTTGGGAGGGACCTGGGATGGAGGTGGGGACCACAGACTCAAGATGCTCCACCGAGGCGAGTGGA
TAACCAGGCCCTCCCCTCCCCAAACCCAGGAATCAGAGTCCTCAGCCCCTCCTCCCTCAGACCCAGGAGCCCCGGCC
CAGCCCCTCCTCCCTCAGACCCAGCAGCCCCGTGCCCAGCTCCTCCCTCAGACCCGTGGGTTCTCCCCTCTAGGAGG
AGCTCTGCGGGGGCCTCTGGCGGCTGGTCCTGGCACAGCGCTGGATGGAGCGGCTCAAGACTGTCGCTGGGTCCAAG
ATGCAAGGCTTGCTGGAGCGCGTGAACACGGAGATACACTTTGTCACCAAATGTGCCTTTCAGGTCAGCCCTCAACT
TAGGGGACAAGTGAGGGGAGGGAGATGCCTTCCTACGAATTAGAAGTAAAGCTCCACTAGGCCTTATTGGCGATTTG
GACCATAGCCACCCAACGAAGGTAGAGCGAGAAGCGCCACCCTGCAGAGCCCTGTTCCTACAGAACAACACGTCCCC
AGGCACCGGTGATGGGGAGCAGTCTGGTCCCATTCTGGGGCCCCGGTTTCCTAGGCCATGATGAAGGGTGCCACTGA
GGGGTTCTTCCCCCAAAAAAAAACAGGGAGAGAAGGGGTCTCTAAACTGAGGAGGCCGGGCGTGGTGGCTCACTCCT
GTAATCCTAGCACTTTGGGAGGCTGAGGTGGGCGGATCACTTGAGGTCAGGAGTTTGAGACCAGCCTGGCCAACATG
ATGAAATCCCGGCTGTACTAAAAATACAAAAATTAGCCGGGCATGGTGGCTCAGGAGGCTGAGGCACAGGAATCGCT
TGAACCCGGGAGCCAGAGGCTGCAGTGAGCCGAGATCATGCCACTGCACTCCAGCCTGGGAGACAGAGTGAAACTGT
CTCAAAAACAAAACAAACAAACAAAAACCTCTCTCTGAGGGCTGGGTGCAGTGGCTCACACCTGTAATCCCAGCACT
TTCGGAGGCCAAGGTGGGAGGATTATTTGAGCCCAGGAGTTCAAGACCAGCCCGGGTAACACAGTGAAACCTCATCT
CTGCACAAAAATAAAAATAAATTAGCCAGGCATGGTGGTGCCCACCTGTGGTCCCAGCTACTCAGAAGGCTGAGGTG
GGAGGATCACTTGAGCCCTGGAGGTCGAGGCTGCGATGAGCTATGATTGGGCCACTGCACTCCAGCTTGAGCGACAG
AGCAAGACCCTGTCTCAAAACATAGAATAGGCCGTGTATGGTGGCTCACGACTGTAATCCCAGCACTTTGGGAGGCT
GAGGCGGGTGGATTGCCTGAGCTCAGGAGTTCGAGACCAGCCTCGGCAACGTGATGAAAACCATCTCTACTAAAATA
CAAAAACAAAATTAGCCAGGCATGGTGGTGGGCACCTGTAGTCCCAGCTACTTGGGAGACTGAGGCAGGAGAATTGC
TTGAACCCAGGAGGCAGAGGTTGCAGTGAGCCGAGATCACACCACTGCCCTCCAGCCTGGGCGACAGAGCAAGACTC
CATCTCCAAAAAAATAAAAATAAAATAAAAGCCTGGGTACAGTGGCTCACGCCTGTAATCCCAGCACTTTGGGAGCC
CGAGGCGGGCAGATCACGAGGTCAGGAGTTTGAGACCACCCTGGCCAATGTGGTGAAACCCCGTCTCTACTAAAAAT
ACAAAAATTAGCTGGGCATGGTGGCGCGCGCCTGTAGTCCCAGCTACTCAGGAGGCTGAGGCAGAATTCCTTGAACC
CGGGAGGTAGAAGTTGCAGTAAGCCGAGATCGTGCCACTGTACTCCAGCCAGGGTGACAGAGCAAGACTCTGTCCCC
AAAAAATAAATAAATAATAAAGTAACTTTGGGAGGCCGAGGCGGGCGAGTCACCTGAGGTCAGGAGTTCGAGACCAG
CCTGGCCAACATAGAGAAACCCCGTTTCTACTAAAAAATATAAATAAATAAATAAATAAATAAATAGGCTGGGCACG
GTGGCTCACGCCTGTAATCCCACACTTTGGGAGGCCGAGGCGGGCGGATCACAAGGTCAGGAGATCAAGACCATCCT
GGCTAACACAATGAAACCCCATCTCTACTAAAAATACAAACAATTAGCCGGGCGTGGTGGCAGGCGCCTGTAGTCCC
AACTACCTGGGAGGCTGAGGCAGGTGAATGGCGTGAAACCAGGAGGCGGAGCTTGCAGTGAGCCGAGATTGCGCCAC
TGTACTCCAGCCTGGGCAACAGAGCGAGACTCTGTCTTAAAAATAAATAAATAAATAAATATGGCCAAGCACGGTGG
CTCATGCCTACAATCCCAGCACTTTGGGAGGCCAAGGTGGGCAGATCACCTGAGGTCAGGAGTTCGAGACCAGCCTG
GCCAACATGGAGAAACCCCGTCTCTACTACAAATACAAAAAATAGCTGGGCATGGTGGTGGGCACCTATAGTCCCAA
CCACTGAGGAGGCTGAGGCAGGAGAATCACTTGAACCTGGGAGGCAGAGGTTGCATTTCACCACTCCAGCCTGGGCA
ACAGAGTGAGACTCTATCTCAAAAAAAAATTAATTAATTAAATAAATAAACTCTGAGAGCCAGAGCTCACTGGGCCC
TGTTGCTGGGCATCGCCAGCAGGGAAGGGCCTTTGGCCTGCGGAGGGGCGGTGGGGGGATGACGTGGTGGTGACGTC
TCCCTCCCCTGCTCCCAGCCCCCCCCCAGCTGTCTTCGCTTCGTCCAGACCAACATCTCCCGCCTCCTGCAGGAGAC
CTCCGAGCAGCTGGTGGCGCTGAAGCCCTGGATCACTCGCCAGAACTTCTCCCGGTGCCTGGAGCTGCAGTGTCAGC
CCGGTAAAGGCTTCCAGGCACCCCCACTCCTTCCCCTCCTGTCCTCACGGCCGCTCCTCCTCTCTGCACAGTGCATC
CCAGACCCCATCTTTCTCATATTGGTTGTGACAAGGGCAAGCTTATTCCTCTTTCTGGAGCTCAGTTTACCAATTTT
TTTTTTTTTTTTTTGAGACGGAGTCTCGCACTGTCGCCCAGGCTGGAGTGCAGTGGCGTGATCTTGGCTCACTGCAA
GCTCCGCCTCTCGGGTTCATGCCATTCTCCTGCCTCAGCCTCCCAAGTAGCTGGGACCACAGGCGCCCGCCACCACA
CCCGGCTAATTTTTTGTATTTTTAGTAGAGACGGGGTTTTACCGAGTTAAACCAGGATGGTCTCGATCTCCTGACCT
TGTGATCCACCTGCCTTGGCCTCCCAAAGTGCTGGGATTACAGGCGTGAGCCACAGTGCCCAGCCTACCTTTTTTTT
TTTTTTTTTTTTTGAGATGGAGTCTTGCTCTGTCCCCCAGGCTGGAGTGCAGGGGTGCTATCTCGGCTCACTGCAAG
CTCTGCCTCCTGGGTTCACGCCATTCTCTTGCCTCAGCCTCCCCAGCAGCTGGGACTACAGGCGCCTGCCACCTCAC
GCGGCTAATTTTTTTTTTTGTATTTTTAGTAGAGACGGTGTTTCACCGTGTCAGCCAGGATGGTCTCGATCTCCTGA
CCTCGTGATCTGCCCGCCTCGGCCTCCCAAAGTGCTAGGATTACAGATGTGAGCCACCGCGCCCAGCCTATTGTTTT
TTTTTTCTAAGTCGGAGTCTTGCTCTGTCGCCCAGGCTGGAGTGCAGTGGCGCAATCTTGGCTCACTGCAACCTCCA
TCTCCAGGGTTCAAGCGAATCTTCTGCCTCAGCCTTCCGAGTACCTGGGATTACAGATGCGCATACCATGCCTGGCT
AGTTTTTGTGTTTTTAGTAGAGATGGGTTTTCACCATGTTGGCCAGGCTGGTCTTGAACTCCTGACCTCAAGTGATC
CACCCACCTTGGCCTCCCAAAGTGCTGGGATTATAGGCGTGAGCCACCGCGCTCAGCCTATTCACTCATTTAATTTG
TGACAGTCTGATGAGGTAGGTACAATGATTATCCTAGTTTTACAGATGAGCAAACTGAGGCACAGAGAGGCCAAGCA
GCCCATCCAAGGTCACACAGCCAGTGGCAGCCAGGCCTCTCTTTCCTTCCTTACCCCAGCCCTTCTCCTTGGTCACC
CAGCCTCCTCTTTCTCCCCAGACTCCTCAACCCTGCCACCCCCATGGAGTCCCCGGCCCCTGGAGGCCACAGCCCCG
ACAGCCCCGCAGCCCCCTCTGCTCCTCCTACTGCTGCTGCCCGTGGGCCTCCTGCTGCTGGCCGCTGCCTGGTGCCT
GCACTGGCAGAGGACGCGGCGGAGGACACCCCGCCCTGGGGAGCAGGTGAGCAGGCTGGGAAGAGGGGGTGAGGGGG
CCGAGAGGGTGGCCCACTTGTGGCTGACACTTTGGGGCCCACAGGTGCCCCCCGTCCCCAGTCCCCAGGACCTGCTG
CTTGTGGAGCACTGACCTGGCCAAGGCCTCATCCTGGTGAGTCCTTCCTGGGCTATGGGGCCTGGACTTTGTGTCTG
CAGGTTGGGAGGGTCACTAGGAGGCCATGGAAGGGTGGTGAGCAGTGGAGGGGCAGGGGCAGCTCTAGGTGCAGAAA
GACCCCTCTGGGGCCAGGCGCGGTGGCTCACGCCTGTAATCCCAGCACTTTGGGAGGCCGAGGCGGGTGGATCACTT
GAGGTCAGGAGTTCAAGACCAGCCTGGCCAACATGGCAAACCCCAGTCTCTACTAAAAATACAAAAATTAACTGGGC
ATAGTGGTGTGTGCCTGTAATCCCAGCTACTCAGGAGGCTGAGGAAGGAGAATTGCTTGAACCCAGGAGGCGGAGGT
TGCAGTGAGCCGAGATTGCGCCACTGGACTCCAGCCTGAGTGACAGAGCTAGACTCCGTCTCAAAAAAAAAAAAAAA
AAAGAAAAGGAAAGACCCCTCTGGGGCCACGGAGACAGACTGGAGACCAAGCCGGAGGCTGTGGGAAGGCCCAGGGT
GAGGACCATGACTGAGCCAGGGCTGGGACCATGGGATGGAGTGGAGTGGATGGGCAGAGAGTCAGGGGTCAGAGATC
AGGAGGGACCGGCCTGGGGCCTGACAGGCTTGAGTTGGCACAGATAGCCCTGGTCCAGTGATGGGGTGGACAGGGGG
GAACCTGGGGGAGGAGTCAGATTAGGGAAAGACATGGAGCTCACTGGGGACCTGAAGGAGCAAGGGGCCCTGGGGAG
ATCTGTGGATGTGGGAGTTTGCAAGCCTCCTGGGACAGGAAGGAAATGGCATTCCAGAAAGAGGGAACTGCCTTTGG
AAGCCCTTTGGTATGAGAAAGCTCAGGGTGTTCCAGTAACTTGGGCCAGCGTGGGGCAGCAAGGAGCCATGGGAGGT
GTGAGAGGGAGGAAAGGAGCTGGGAACAGGTGTGGAGGGTGCAGGCAGGTAGGCAGGTGGGCCAGGAGGGAAGAAGG
AGGGGAGGCCCCAGCGGGTCCCCTAACATTTCAAATCTAACATTTTGTAGTAATTGCTCGCACATATAGTGCCTTGG
TAATGCAGGCCTCATGTGGGGCTGGCTTGCTTGCTTTATTTTTTTTTGAGATGGACTCTCACTCTGTCACCCAGGCT
GGAGTGCAGTGGCGGGATCTTGGCTCACTGCAACCTCCACCTCCCAGGTTCAAGCAATTCTCCTGCCTCAGCCTCCC
AAGTAGCTGGGATTACAGGCGCCCGCCACCATGCCTGGCTAATTTTTTGTATTTTTAGTAGAGATGGGGTTTCGCCA
TGTTGGCCAGGCTGGTCTCAAACTCCTGATCTCAGCTGATCCGCCCGCCTCAGCCTCCCAAAGTGCTGGGATGACAG
GTGTGAGCCACTGTTCCTGGCCCATGCTGGGCTTTCCACAAATGAACTCACTTCCTTGAGAAGTTCGTGCCATTATG
ATCCCAAGGCATAGAGAGGTAAATAATTTGCCAAAAGTCACACAGCTCAAGGGTGACAACCAAGATTTGACTTCAGG
TTGTCTTTTTTTTTTTTTTGAGATGGAGTCTTGCTCTGTCGCCCAGGCTGTAGTGCAGTGGCATGATCTCGGCTCAC
TGCAGCCCCCGCCTCCCAGGTTCAAGTGATTCTCCTGCCTCAGCCTCTGGAGTAGCTGGGATTACAGGTACCCACCA
CCACACTCGGCTAATTTTTGTATTTGTAGTAGAGACGGGGTTTCACCATGTTGGCCAGGCTGGTCTCGAACTCCTGA
CCTTAGCTGATCCGCCCGCCTCAGCCTCCCAAAGTGCTGGGATTACAGGCATGAGCCACCACGCCCGGCCTCGAGTT
TGTCTTGATCCAGCATCTGTGCCCTTCACTCTGCTGCCTCAGTTTCCTGATCTGTACAAAAGGCTCTAATAATAGTC
CTTCCACCATAGGGTTGTTATGGGGTGAAGTGAAGTGACACAGGCAAGGCTCTTAGAGCAACGCAGTGGAAACGGCA
TGTGAGCCACCTGTGCGATTTTCACTTTCCTGGTAGCCACATTTTTTTTTTTTTTTTCCTGAGATGGAGTCTCACTC
TGTCGCCAGGTTGAAGTGCAGTGGTGTGATCTCAGCTCACTGCAACCTCCACCTCCCGGGTTCAAGCGATTCTCCTG
CTTCAGTCTACCGAGTAGCTGGGATTACAGGCACGCGCCATCACGCCCGGCTAATTTTTTGTGTTTTTAGTAGAGAC
AAGGTTTCACCATGTTGGTCAGGCTGGTCTCGAACTCCTGACCTCATGATCCACCCGCCTCAGCCTCCCAAAGTGCT
GGGATTACAGGCGTGAGCCACCGCGCCCGGGGTCACAGTTTTTAAAAAAGGAAAAAGAAGCAGGTAACTTTATTTTA
TGCAGTATATCCAAAATGTTGTTATTTCAACATGTAATCAGTATTTCTCAATGATTAATGAGACATTAATCAGTTGG
AATCATTCATCATTTTGGGATATTTTACATTCTTTTGTCATACTAAGCCTTCAGCATCCAACGTATAGTTTATTTAT
TTATTTATTTTTCCTTTTGAGATGGATTCTCGCTCTGTCACCCAGGCTGGAGTGCAGTGGCGCGATCTCAGCTCACT
GCAACCTCGGACTCCCTGGTTCAGGCGATTCTCCTGCCTCAGCCTCCCGAGTACCTGGGATTACGCCACCACACCCA
GCTAATTTTTGTACTTTTAGTAGAGACCGGGTTTCACCATGTTGGCCAGGATGATCTCCATCCCCTGACCTCGTGAT
CCGCCCGCCTCAGCCTCCCAAAGTGCTGGATTACAAGTGTAAGCCACCGCGCCCAGCCCAGACAATAAATTTTTATC
AGAAATGCTTCATCTATATTTAAATTTCATGAAATTTACAGTTGCCAAAAGTAGACTCACATAGTCAAGTTGTTCAA
AACATACATTAAACCAGCCTGGGCCACATGGTGAAACCCCATCTCTACAAAAAATTTTAAAAAGTAGCTGGGTGTGG
TGGCACATGCCTGCAGTCCCAGCTACTTAGGAGGCAAAGTAGGAGGATTGCTTGAGCCCAGGAGGTCGAGGCTGCAG
TGAGCCCTGATTGTGCCACTGCACTCCAGCCTGGGTGAGAGAGCAAGACCCTGTCTCAAAAACAAAAAAACCACACA
GAAGAGTTTAGAATCCCAGTTGCTATCTCTGGGAATCATCATGATTGCTAAGACCGGTATCTATCCAACAACAACAA
AAATAATTAGTGCACTCCAGCCTGGGCAATAGAGGGAGACTCTGTCTCAAAAAAAAAAAAAAAAAAAAAAAAAAGAT
CAAAGACCAGTATCTAAATTGCTCTGTCGCCCAGGGTGGAGTGCAGTGGCACGATCTCGGCTCACTGCAACCTCCGC
CTCCCGGGTTCAAGTGATTCTCCTGCCTCAGCCTCCCCAGTAGCTTGGACTACAGGCGCGTGCCACCACACCCAGCT
AATTTTTGTATTTTTAGTAGAGGTAGGGTTTCACTATGTTTGCCAGGATTGTCTCGATCTCTTGACCTCGTGATCTG
CCCGCCTCAGCCTCCCAAAGTGCTGGGATTATAGGCGTGAGCCACCACGCCCAGCCTATCTTTTTTTTTTTTTTTAA
GAAAAGGTAATACATTGAGCAGTTTTCCTCCCAACCTAGTCCCTGTCCAATCTGTTTCCTGTCTTAAATTTATTTTT
TATTATAATTTTTTTTTTTTTTTTTTTTTGTGAGACAGACTTTTGCTCTTGTTAGCCAGGCTGGAGTGCAGTGGTGC
TATCTTGACTCACGGCAACCTCCACCTCCCAGGTTCAAGCGATTCTCCTGCCTCAGCCTCTGGAGTAGCTTGGATTA
CAAGCGCCTGCCACCACGCCCAACTAATTTAGTGTATTTTTAGTAGAGACGGGGTTTCTTTTGTTGTTGTTGTTGTT
TTTGAGACGGAGTCTCACTCTATTGCCCAGGCTGGAGTGCAACGGCACGATCTCGGCTCACTGCAACATCTGCCTCC
CGGGTTCAAGCGATTCTACTGCCTCAGCCTCCTGAGTAGCTGGGATTACAGGCACACACCACCACACCTGGCTAATT
TTTGTATTTTTAGTAGAGATGGGGTTTCACCATATTGGCCAGGCTGATCTCGAACTCCTGACCTCGTGATCTGCCCG
CCTTGACCTCCCAAAGTGATAGGATTACAGCCATGAGCCGCCCTGCCCAACTAATACCTGATATTAATTTTATTGTA
TCTCACCCAGCATGGTGGCTCACACCTGTAACTCCTTGGCAGATCGCTTGAGCCTAAGAGTTCAAGACCAGCTTGGG
CAACATGGTGAAACCCCGTCTCTACTAAAAATATAAAAATGAGCTGGGCATGGTGGCGTGCACCCGTAATCCCAGCT
AACTCAGGAGGCTGAGGCAGGAAGAATGGCTTGAACCCGGGAGGCAGAGGCTGCAGTGAGCTGAGATCATGCCACTG
CATTCCAGCCTGAAACAGAGCAAGACTGTCTCAAAAGAAGTTTAAGTCCAGCCTCAAACTCCTGGACTCAAGTGATC
CTCCTGCCTCAGCCTCCTGAGTAGTGCACCGGCCCCATCTCAAGTGTTCAGTATTCACATGTGGCTTGTTACTCCCG
TATTAAAGAGCAAAAATAGAGAACGTTTCTTTTTCTTTTTTTTCTTTTTTTTTTTTTTTTTGAGATGGGAGTTTCGC
TCTTGTCGCCCAGGCTGGAGTGCAATGGCACGATCTTGGCTCACCGCAACCTCTGCCTCCCAGGTTCAAGCGATTCT
CCTGCCTCAGCCTCCCAAGTAGCTGGGATTACAGACATGCACCACCATGCCTAGCAAATTTTGTTTTTGTTTTTGTT
TTTTTCTTTTGAGACAGACTTTTGCTTTTATTGCCAAGGCTGGAGTGCAATGGTGCAATCTCGACTCACTGCAACCT
CCGCCTCCCGGGTTCAAGCAATTCTCCTGCCTCAGCTAATTTTGTAGTTTTAGTAGAAACGGTTTCTCCATGTTAGC
TAGGCTGGTCTCAAACTCCCAACCTCAGGTGATCCGCCCGCCTCGACCTCCTAAAGTGCTGGAATTACAGGCGTGAG
CCACTGTGCCTGGCCCTAATTTTGTATTTTTAGTAGAGATGGGGTTTCTCCCTGTTGGTCAGGCTGGTCTCAAACTC
CCAACCTAAGGTGATCCACCAGCCTCAGCCTCCCAAACTGCTGGGATTATAGGCATGAGCCACCGCGCCCGGGGAAA
TATAGGGAACATTTCTATCATGGCCACGTTCTCTTGGACAGCAATGCCCTAGCACATAGTAAGCGCTTGCTGGGAAT
GGAGTTGTTTTGGGCATAAATCTGTGGCCCGCCCCAAGCTAAGCTACTTTCCCTCTCCTCTCTCAGCGGAGCCTTAA
ACAACGCAGTGAGACAGACATCTATCATCCCATTTTACAGGGGAGGATACTGAGGCACACAGAGGGGAGTCACCAGC
CAGAGGATGCATAGCCTGGACACAGAGGAAGTTGGCTAGAGGCCGGTCCCTTCCTTGGGCCCCTCTCATTCCCTCCC
CAGAATGGAGGCAACGCCAGAATCCAGCACCGGCCCCATTTACCCAACTCTGTACAAAGCCCTTGTCCCCATGAAAT
TGTATATAAATCATCCTTTTCTACCAGCTCTGGCCAGGTCTGTCTATGGATGGGTGTGAATGGGGTAGTTTTGATTT
CAGAATCTTGGTTTTTGAATTGCAATGCAATGTTTCATTCAATACGTGTTTTCTGAGGTCCCCTGGAGCCCCGGGCC
GGCGCTGGGCAATGTCAGAGCCTGGATTTGAGTCAGAACCAGCCCAGGTGCCCCAGGTCTCCTTGGCTATGGACGAG
AAGACAAGGGATTCAGCGAGCCTGGGTGCAGGATGCCATAGGCTACTATGGAGATAAAACAGAGGCAGTGTGAGGGG
CAACTCACCCAGGCAGGGAGAGCAGAGAGGCTTTCCAGAGCCAAATGGCATTGCGATGGTGACAAGCCAGATTTGGA
GGGGGTGGGGAGGGCATCCTGGGCAAGGGACCCAGCTGAAGCAAAGGCCGACAGTCCAGAAAGTGAGAAGTTCCTCT
GAGGAACGCTCAGTGGATTGGTGCAGCCAGGCAACTAGAAGCCATTCCTTGATCCACTCTTAGAGCCCCTGGGGAAG
ACCACCTCCATCTGTTTTACAGATGGATAAACTGAGGCCCAGATAAGGCCAAGGGTTTCTCCAGGTCACAGTGAAGT
CGACCAGACACAGGAATCCAAATCTCTAAGCCTAGAATGGGCAAGGCTCCGGGGCTCACGCCGGTAATCCCAGCACT
TTGGGAGGCCGAAACGGGAGGATCGCTTGAGCCCAGGAGTTCGAGATCAGCCTGGCCAACATGTCAGACTCCCCCCG
TTCCCGGGCCACAACAAAATATATATATATATATATATTAGCCAGGCATGGTGGCGCGCCCCTGTAGTCCAAGTTAC
TCGAGAGGCTGAGGCAGGAG (SEQ ID NO: 7)

### REFERENCES

1. P. Tsapogas, C. J. Mooney, G. Brown, A. Rolink, The Cytokine Flt3-Ligand in Normal and Malignant Hematopoiesis. International journal of molecular sciences 18, (2017).
2. C. J. Mooney, A. Cunningham, P. Tsapogas, K. M. Toellner, G. Brown, Selective Expression of Flt3 within the Mouse Hematopoietic Stem Cell Compartment. International journal of molecular sciences 18, (2017).
3. L. D. Shultz, M. A. Brehm, J. V. Garcia-Martinez, D. L. Greiner, Humanized mice for immune system investigation: progress, promise and challenges. Nat Rev Immunol 12, 786-798 (2012).
4. K. Palucka, J. Banchereau, Cancer immunotherapy via dendritic cells. Nature reviews. Cancer 12, 265-277 (2012).
5. J. S. Do, B. Min, IL-15 produced and trans-presented by DCs underlies homeostatic competition between CD8 and {gamma} {delta} T cells in vivo. Blood 113, 6361-6371 (2009).
6. J. C. Oliveros et al., Breaking-Cas-interactive design of guide RNAs for CRISPR-Cas experiments for ENSEMBL genomes. Nucleic acids research 44, W267-271 (2016).
7. P. D. Hsu et al., DNA targeting specificity of RNA-guided Cas9 nucleases. Nature biotechnology 31, 827-832 (2013).
8. Y. Fu, J. D. Sander, D. Reyon, V. M. Cascio, J. K. Joung, Improving CRISPR-Cas nuclease specificity using truncated guide RNAs. Nature biotechnology 32, 279-284 (2014).
9. B. E. Low, P. M. Kutny, M. V. Wiles, Simple, Efficient CRISPR-Cas9-Mediated Gene Editing in Mice: Strategies and Methods. Methods Mol Biol 1438, 19-53 (2016).

All references, patents and patent applications disclosed herein are incorporated by reference with respect to the subject matter for which each is cited, which in some cases may encompass the entirety of the document.

The indefinite articles “a” and “an,” as used herein the specification and in the claims, unless clearly indicated to the contrary, should be understood to mean “at least one.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

The terms “about” and “substantially” preceding a numerical value mean ±10% of the recited numerical value.

Where a range of values is provided, each value between the upper and lower ends of the range are specifically contemplated and described herein.

1.-55. (canceled)

56. A method for evaluating an effect of an agent on human immune cells in a transgenic mouse, the method comprising:
administering an agent to a transgenic mouse whose genome comprises a human FLT3L transgene and a genomically inactivated mouse Flt3 allele, wherein the transgenic mouse has been engrafted with human immune cells; and
evaluating an effect of the agent on the human immune cells.

57. The method of claim 56, wherein the human immune cells are selected from T cells, dendritic cells, natural killer cells, and macrophages.

58. The method of claim 56, wherein the agent is a therapeutic agent.

59. The method of claim 56, wherein the agent is an anti-cancer agent.

60. The method of claim 56, wherein the agent is an anti-inflammatory agent.

61. The method of claim 56, wherein the agent is a drug.

62. The method of claim 56, wherein the agent is an immunogenic composition.

63. The method of claim 56, wherein the transgenic mouse comprises human tumor cells.

64. The method of claim 63, wherein evaluating the effect of the agent on the human immune cells comprises measuring a human tumor cell response.

65. The method of claim 64, wherein the human tumor cell response is selected from cell death and cell proliferation.

66. The method of claim 56, wherein evaluating the effect of the agent on the human immune cells comprises measuring a human immune cell response.

67. The method of claim 66, wherein the human immune cell response is cell signaling.

68. The method of claim 56, wherein the transgenic mouse has a non-obese diabetic (NOD) background.

69. The method of claim 68, wherein the transgenic mouse has Il2rg^{tm1Wj1} mutation.

70. The method of claim 69, wherein the transgenic mouse has a Prkdc^{scid} mutation.

71. The method of claim 56, wherein the genomically inactivated mouse Flt3 allele comprises a genomic modification in exon 6, exon 7, and/or exon 8 of the mouse Flt3 allele.

72. The method of claim 71, wherein the genomic modification is a genomic deletion.

73. The method of claim 72, wherein the nucleic acid sequence of SEQ ID NO: 5 has been deleted from the mouse Flt3 allele.

74. The method of claim 73, wherein the genomically inactivated mouse Flt3 allele comprises the nucleic acid sequence of SEQ ID NO: 6.

75. The method of claim 56, wherein the human FLT3L transgene comprises a nucleic acid sequence of SEQ ID NO: 7.


SEQUENCE LISTING (7 sequences)

SEQ ID NO: 1 - RNA, 19 nt, synthetic construct
  misc_feature 1..19: note = Synthetic polynucleotide
  source 1..19: mol_type = other RNA
gaacagcttggtgcattcg

SEQ ID NO: 2 - RNA, 19 nt, synthetic construct
  misc_feature 1..19: note = Synthetic polynucleotide
  source 1..19: mol_type = other RNA
gatggtcaccaacgctgac

SEQ ID NO: 3 - DNA, 24 bp, synthetic construct
  misc_feature 1..24: note = Synthetic polynucleotide
  source 1..24: mol_type = other DNA
ccaacctgaactgtatggagatag

SEQ ID NO: 4 - DNA, 20 bp, synthetic construct
  misc_feature 1..20: note = Synthetic polynucleotide
  source 1..20: mol_type = other DNA
ccacagggaaagccactaaa

SEQ ID NO: 5 - DNA, 1500 bp, Mus musculus
  source 1..1500: mol_type = genomic DNA
atcccccagtgctggtgaccttgaacacagggtctgccctgagcagtgctgacagcctgttagctgtcattttgaagcatttcctgaaggaaacttaccttatcattgcagctgtaaagaagaaggccctgctgttgtcagaaaggaggaaaaggtacttcatgagttgttcggaacagacatcagatgctgtgctagaaatgcactgggccgcgaatgcaccaagctgttcaccataggtaatgggggactgtcggctatgtgtccttagtgacggttcctaagggaccggtgatagcttctgtgtgggtaatcattccacttcagaaagatgatggaaagttatactgaaatccgaatctagatcgaatagtccacattctgacataaaagcaattgaacacacaggtatggccaagcttggtggcccgggcttttaatccttctttcataggactttgatttgattcaatttgatttgatttgatttgacttaggtgctgggctcaaacccagggactcagagcatctgctttgccactgagttccactccagatcttttgcaccactttatttatttatttattgtgtgtgtgtatacacacattaaatacacacacatacacacacacacacacacacacacacacacacacacacacacacacacacgctacagcatgtaatgtggatgtcaggttctcttcttgtcatatggggcccctggatcaaattcaggtcagtctggcagcggcaggcacctccacccactgagccatcttgatgccccgttcataccatttacaatgtaacactgtagattctgtaagggaggatttctgcaacaaagggtattccatctcgagtgggtggttttactctgtcgcttttttttttttagttttgtgctgctctgtgactccaggtcttctccaggccagcctcttttaccctgatcttctcagctctgcattgtttggcttgattagtgatggcagggatgctgacaccattgcttttcaaaacgtttgcgacacttctgagctgaggcaaaagtttttaaaaaaaagatgtgcttttttttttttttttttgtcttctttgggatatttttatcttctagatctaaaccaggctcctcagagcacactgccccagttattcctgaaagtgggggaacccttgtggatcaggtgtaaggccatccatgtgaaccatggattcgggctcacctgggagctggaagacaaagccctggaggaggtaacagcgcccacccgtggtgtttaccgtaagctctcagcgtgggctggaaacccggaatacagactcccctttcattaatactcagggcagctactttgagatgagtacctactccacaaacaggaccatgattcggattctcttggcctttgtgtcttccgtgggaaggaacgacaccggatattacacctgctcttcctcaaagcaccccagccagtcagcgttggtgacca

SEQ ID NO: 6 - DNA, 442 bp, synthetic construct
  misc_feature 1..442: note = Synthetic polynucleotide
  source 1..442: mol_type = other DNA
ccaacctgaactgtatggagatagcttgtctatggggaaaaaaaatcaccaaataaaataaaatctaaaatattaaataataaagtaatactgtaacattatcctaattagatacagagtagctagctggtccctgcagattccaggctggatacagttgagttaatttttcatctgctcttttcttccatccctaaaaatatactttgtaacttcatcccctaaacatactgggtccccccaccccatcctaggtaacgggctcacgaaatttggggttggttggtttgtttgtttttaatgctgtcctggaactcgctgtgtagattggttggccttgaactcacagaaatctaccagcatctgcctccccagtgctgggattaaaggcacaggccaccaagcttgactctgcttgtgtctttagtggctttccctgtgg

SEQ ID NO: 7 - DNA, 13880 bp, Homo sapiens
  source 1..13880: mol_type = genomic DNA
gatccctgtgctcctccgagtggcacaggaaacttggctgatagaggcgctctctccatgcatcctccaccatccactccccggtcccttagacttccattcagaagtgacacctgtcacctccaccctcatgtgactggccaaagtgagtgtggccatgtccgagttccccaggatgggacctcgaatcctcctgctgggggggtgggggcagcagatgtttcaggacagtctgccacggatcagatgggtgagcaagagattcaggatgggcgtgatgggtcacacctgtaatcccagcattttgggaggccgaggcaggctgatcacctgaggtcaggagttcgagatcagcctggccaacatagtgaaacctcgtctctactaaaaatacaaaaattagccgggcatggtggcacgcacctgcaattccagctacttgggagcctgaggcacaagaattgcttaagcccaggaggcggaggttgccgtgagccgagactgtgccactgcactccagcctgggcaacagagtgagaccccatctaaaaaaaaaaaatgcttcagagattcagagatccacagatgtggaagagagcttctattttgtctcatgtaccacctcacccctcagtttggagttggagaagccgaggcttagagaggtcaaggggctggccaagctcccacagtgataaagctagtgctagggcgacacctgccaggcccaatgtgacaagatgcatcctgagtcctgtgtgccaagccttggcggggcaaagttggggcccggaggacggtaagacacagcaactgcgagttcccagagacagatccgggttccagccccagctcaccatgcactgggcgtgccacccagggagtcagccccactgcagcccccgctgcggggcgtccggagaacgcgccatctgcggcgtgagcggccgcctctcaccaccagggggcgcgctccgcctgggcccagattccaccccttgactgtctccccccaaaaatttcctttcactttcggtctctggctgtcacccggcttggccccttccacacccaactggggcaagcctggtgcgtgaggagacttggactctaggtccccaaggggcggagccagggtccccactcctggggcagggggagcagagggtggggggctgggttcccgggtccctaacctggggagggttctggggaccagacgtcgaggttcttagaagtggagatgaagagttttcaccgtaactggggcagacgcaggaagcctgggggaggaaggggcggaaactcagccacatcaatgggacgcgggaggggcggggaggcaaaggggcgggcagggcaggggctggggcatgagggtccgagacttgttcttctgtcccttccaagacccggcgacaggaggcatgaggggcccccggccgaaatgacagtgctggcgccagcctggagcccaacagtgcgtaaaccccagggacaagatcaggggagaggggaggcacaatgtcaggatggggcagagatgaggggagatggacgggagaacagatggacagatgacgaggaaataggaggggagatggacagatgtgaggggagatggataggagaggagacggacagaggagggggagatggacagaggatggggagatggacagaggagggggagatagaggagagggagatagagaggagtgggagatggacaggagcggggagatggacagaggagggggagatggacagaggagagggcagatgaacagaggagagggagataaagaggagggaggtggacaggaggagggagatggacagagaagggggagatggacagaggtgggggagatggacaggagggaagatggagaggagggggtatggacagaggagaggggagatggacaggagggggagatggacagaggagagggagatgaagaggagggggaggtggacaggaggagggagacggacagaggagaggaagagagacagaggaggggggagatggacagaggagggggagatggacagaaaaggggggagatggacagaggagagggaaggtggacagagccaagaacaaatgaagaggacgtggaccaagatcaagagagaagcaggcaacagtggtgtagaaggcagagggagggacagagctggaggaacccgggcgaggaaaccagacgtgaagatgaggtggttggagagagaccagcagagtgggggagatggggagagagaggggtggggcagagggggatgcaaactggacagcattggaccagaggcagagagaaaccgggaaagacaggcagagatggggccatgtctccagaaagtgtggaagaggcagaaggacacccagggaaggaggagcggggaagacagaacagtacaggtgggaagcccggaggagggggctgtgtgtggaacagcagagggctcccccagcacccgctcccctgcagacctatctcctcctgctgctgctgctgagctcgggactcagtgggacccaggactgctccttccaacacagccccatctcctccgacttcgctgtcaaaatccgtgagctggtgagcggcgctgccccggaccccctcatgtgatcccccttccccccacttttttttttaagtagagatggggtctctctccctgtgtttcccagggtggtcttgaactcctgggctcaagcgatcctcccaccttggcctcccaaagtgctggaattacaggcgtgagccactgtgcttaggggtgtcatccctttttaagggcaaggttctgtggcttcttctgggctccccctctcttggtcttgtccctctctctctggatctctgctgccacctctgggtccccacagttctgtttctcgctgttttcagccaggcctgatcctgttttctcccgcagtctgactacctgcttcaagattacccagtcaccgtggcctccaacctgcaggacgtaagtcatgttgggagggacctgggatggaggtggggaccacagactcaagatgctccaccgaggcgagtggataaccaggccctcccctccccaaacccaggaatcagagtcctcagcccctcctccctcagacccaggagccccggcccagcccctcctccctcagacccagcagccccgtgcccagctcctccctcagacccgtgggttctcccctctaggaggagctctgcgggggcctctggcggctggtcctggcacagcgctggatggagcggctcaagactgtcgctgggtccaagatgcaaggcttgctggagcgcgtgaacacggagatacactttgtcaccaaatgtgcctttcaggtcagccctcaacttaggggacaagtgaggggagggagatgccttcctacgaattagaagtaaagctccactaggccttattggcgatttggaccatagccacccaacgaaggtagagcgagaagcgccaccctgcagagccctgttcctacagaacaacacgtccccaggcaccggtgatggggagcagtctggtcccattctggggccccggtttcctaggccatgatgaagggtgccactgaggggttcttcccccaaaaaaaaacagggagagaaggggtctctaaactgaggaggccgggcgtggtggctcactcctgtaatcctagcactttgggaggctgaggtgggcggatcacttgaggtcaggagtttgagaccagcctggccaacatgatgaaatcccggctgtactaaaaatacaaaaattagccgggcatggtggctcaggaggctgaggcacaggaatcgcttgaacccgggagccagaggctgcagtgagccgagatcatgccactgcactccagcctgggagacagagtgaaactgtctcaaaaacaaaacaaacaaacaaaaacctctctctgagggctgggtgcagtggctcacacctgtaatcccagcactttcggaggccaaggtgggaggattatttgagcccaggagttcaagaccagcccgggtaacacagtgaaacctcatctctgcacaaaaataaaaataaattagccaggcatggtggtgcccacctgtggtcccagctactcagaaggctgaggtgggaggatcacttgagccctggaggtcgaggctgcgatgagctatgattgggccactgcactccagcttgagcgacagagcaagaccctgtctcaaaacatagaataggccgtgtatggtggctcacgactgtaatcccagcactttgggaggctgaggcgggtggattgcctgagctcaggagttcgagaccagcctcggcaacgtgatgaaaaccatctctactaaaatacaaaaacaaaattagccaggcatggtggtgggcacctgtagtcccagctacttgggagactgaggcaggagaattgcttgaacccaggaggcagaggttgcagtgagccgagatcacaccactgccctccagcctgggcgacagagcaagactccatctccaaaaaaataaaaataaaataaaagcctgggtacagtggctcacgcctgtaatcccagcactttgggagcccgaggcgggcagatcacgaggtcaggagtttgagaccaccctggccaatgtggtgaaaccccgtctctactaaaaatacaaaaattagctgggcatggtggcgcgcgcctgtagtcccagctactcaggaggctgaggcagaattccttgaacccgggaggtagaagttgcagtaagccgagatcgtgccactgtactccagccagggtgacagagcaagactctgtccccaaaaaataaataaataataaagtaactttgggaggccgaggcgggcgagtcacctgaggtcaggagttcgagaccagcctggccaacatagagaaaccccgtttctactaaaaaatataaataaataaataaataaataaataggctgggcacggtggctcacgcctgtaatcccacactttgggaggccgaggcgggcggatcacaaggtcaggagatcaagaccatcctggctaacacaatgaaaccccatctctactaaaaatacaaacaattagccgggcgtggtggcaggcgcctgtagtcccaactacctgggaggctgaggcaggtgaatggcgtgaaaccaggaggcggagcttgcagtgagccgagattgcgccactgtactccagcctgggcaacagagcgagactctgtcttaaaaataaataaataaataaatatggccaagcacggtggctcatgcctacaatcccagcactttgggaggccaaggtgggcagatcacctgaggtcaggagttcgagaccagcctggccaacatggagaaaccccgtctctactacaaatacaaaaaatagctgggcatggtggtgggcacctatagtcccaaccactgaggaggctgaggcaggagaatcacttgaacctgggaggcagaggttgcatttcaccactccagcctgggcaacagagtgagactctatctcaaaaaaaaattaattaattaaataaataaactctgagagccagagctcactgggccctgttgctgggcatcgccagcagggaagggcctttggcctgcggaggggcggtggggggatgacgtggtggtgacgtctccctcccctgctcccagcccccccccagctgtcttcgcttcgtccagaccaacatctcccgcctcctgcaggagacctccgagcagctggtggcgctgaagccctggatcactcgccagaacttctcccggtgcctggagctgcagtgtcagcccggtaaaggcttccaggcacccccactccttcccctcctgtcctcacggccgctcctcctctctgcacagtgcatcccagaccccatctttctcatattggttgtgacaagggcaagcttattcctctttctggagctcagtttaccaattttttttttttttttttgagacggagtctcgcactgtcgcccaggctggagtgcagtggcgtgatcttggctcactgcaagctccgcctctcgggttcatgccattctcctgcctcagcctcccaagtagctgggaccacaggcgcccgccaccacacccggctaattttttgtatttttagtagagacggggttttaccgagttaaaccaggatggtctcgatctcctgaccttgtgatccacctgccttggcctcccaaagtgctgggattacaggcgtgagccacagtgcccagcctacctttttttttttttttttttttgagatggagtcttgctctgtcccccaggctggagtgcaggggtgctatctcggctcactgcaagctctgcctcctgggttcacgccattctcttgcctcagcctccccagcagctgggactacaggcgcctgccacctcacgcggctaatttttttttttgtatttttagtagagacggtgtttcaccgtgtcagccaggatggtctcgatctcctgacctcgtgatctgcccgcctcggcctcccaaagtgctaggattacagatgtgagccaccgcgcccagcctattgttttttttttctaagtcggagtcttgctctgtcgcccaggctggagtgcagtggcgcaatcttggctcactgcaacctccatctccagggttcaagcgaatcttctgcctcagccttccgagtacctgggattacagatgcgcataccatgcctggctagtttttgtgtttttagtagagatgggttttcaccatgttggccaggctggtcttgaactcctgacctcaagtgatccacccaccttggcctcccaaagtgctgggattataggcgtgagccaccgcgctcagcctattcactcatttaatttgtgacagtctgatgaggtaggtacaatgattatcctagttttacagatgagcaaactgaggcacagagaggccaagcagcccatccaaggtcacacagccagtggcagccaggcctctctttccttccttaccccagcccttctccttggtcacccagcctcctctttctccccagactcctcaaccctgccacccccatggagtccccggcccctggaggccacagccccgacagccccgcagccccctctgctcctcctactgctgctgcccgtgggcctcctgctgctggccgctgcctggtgcctgcactggcagaggacgcggcggaggacaccccgccctggggagcaggtgagcaggctgggaagagggggtgagggggccgagagggtggcccacttgtggctgacactttggggcccacaggtgccccccgtccccagtccccaggacctgctgcttgtggagcactgacctggccaaggcctcatcctggtgagtccttcctgggctatggggcctggactttgtgtctgcaggttgggagggtcactaggaggccatggaagggtggtgagcagtggaggggcaggggcagctctaggtgcagaaagacccctctggggccaggcgcggtggctcacgcctgtaatcccagcactttgggaggccgaggcgggtggatcacttgaggtcaggagttcaagaccagcctggccaacatggcaaaccccagtctctactaaaaatacaaaaattaactgggcatagtggtgtgtgcctgtaatcccagctactcaggaggctgaggaaggagaattgcttgaacccaggaggcggaggttgcagtgagccgagattgcgccactggactccagcctgagtgacagagctagactccgtctcaaaaaaaaaaaaaaaaaagaaaaggaaagacccctctggggccacggagacagactggagaccaagccggaggctgtgggaaggcccagggtgaggaccatgactgagccagggctgggaccatgggatggagtggagtggatgggcagagagtcaggggtcagagatcaggagggaccggcctggggcctgacaggcttgagttggcacagatagccctggtccagtgatggggtggacaggggggaacctgggggaggagtcagattagggaaagacatggagctcactggggacctgaaggagcaaggggccctggggagatctgtggatgtgggagtttgcaagcctcctgggacaggaaggaaatggcattccagaaagagggaactgcctttggaagccctttggtatgagaaagctcagggtgttccagtaacttgggccagcgtggggcagcaaggagccatgggaggtgtgagagggaggaaaggagctgggaacaggtgtggagggtgcaggcaggtaggcaggtgggccaggagggaagaaggaggggaggccccagcgggtcccctaacatttcaaatctaacattttgtagtaattgctcgcacatatagtgccttggtaatgcaggcctcatgtggggctggcttgcttgctttatttttttttgagatggactctcactctgtcacccaggctggagtgcagtggcgggatcttggctcactgcaacctccacctcccaggttcaagcaattctcctgcctcagcctcccaagtagctgggattacaggcgcccgccaccatgcctggctaattttttgtatttttagtagagatggggtttcgccatgttggccaggctggtctcaaactcctgatctcagctgatccgcccgcctcagcctcccaaagtgctgggatgacaggtgtgagccactgttcctggcccatgctgggctttccacaaatgaactcacttccttgagaagttcgtgccattatgatcccaaggcatagagaggtaaataatttgccaaaagtcacacagctcaagggtgacaaccaagatttgacttcaggttgtcttttttttttttttgagatggagtcttgctctgtcgcccaggctgtagtgcagtggcatgatctcggctcactgcagcccccgcctcccaggttcaagtgattctcctgcctcagcctctggagtagctgggattacaggtacccaccaccacactcggctaatttttgtatttgtagtagagacggggtttcaccatgttggccaggctggtctcgaactcctgaccttagctgatccgcccgcctcagcctcccaaagtgctgggattacaggcatgagccaccacgcccggcctcgagtttgtcttgatccagcatctgtgcccttcactctgctgcctcagtttcctgatctgtacaaaaggctctaataatagtccttccaccatagggttgttatggggtgaagtgaagtgacacaggcaaggctcttagagcaacgcagtggaaacggcatgtgagccacctgtgcgattttcactttcctggtagccacattttttttttttttttcctgagatggagtctcactctgtcgccaggttgaagtgcagtggtgtgatctcagctcactgcaacctccacctcccgggttcaagcgattctcctgcttcagtctaccgagtagctgggattacaggcacgcgccatcacgcccggctaattttttgtgtttttagtagagacaaggtttcaccatgttggtcaggctggtctcgaactcctgacctcatgatccacccgcctcagcctcccaaagtgctgggattacaggcgtgagccaccgcgcccggggtcacagtttttaaaaaaggaaaaagaagcaggtaactttattttatgcagtatatccaaaatgttgttatttcaacatgtaatcagtatttctcaatgattaatgagacattaatcagttggaatcattcatcattttgggatattttacattcttttgtcatactaagccttcagcatccaacgtatagtttatttatttatttatttttccttttgagatggattctcgctctgtcacccaggctggagtgcagtggcgcgatctcagctcactgcaacctcggactccctggttcaggcgattctcctgcctcagcctcccgagtacctgggattacgccaccacacccagctaatttttgtacttttagtagagaccgggtttcaccatgttggccaggatgatctccatcccctgacctcgtgatccgcccgcctcagcctcccaaagtgctggattacaagtgtaagccaccgcgcccagcccagacaataaatttttatcagaaatgcttcatctatatttaaatttcatgaaatttacagttgccaaaagtagactcacatagtcaagttgttcaaaacatacattaaaccagcctgggccacatggtgaaaccccatctctacaaaaaattttaaaaagtagctgggtgtggtggcacatgcctgcagtcccagctacttaggaggcaaagtaggaggattgcttgagcccaggaggtcgaggctgcagtgagccctgattgtgccactgcactccagcctgggtgagagagcaagaccctgtctcaaaaacaaaaaaaccacacagaagagtttagaatcccagttgctatctctgggaatcatcatgattgctaagaccggtatctatccaacaacaacaaaaataattagtgcactccagcctgggcaatagagggagactctgtctcaaaaaaaaaaaaaaaaaaaaaaaaaagatcaaagaccagtatctaaattgctctgtcgcccagggtggagtgcagtggcacgatctcggctcactgcaacctccgcctcccgggttcaagtgattctcctgcctcagcctccccagtagcttggactacaggcgcgtgccaccacacccagctaatttttgtatttttagtagaggtagggtttcactatgtttgccaggattgtctcgatctcttgacctcgtgatctgcccgcctcagcctcccaaagtgctgggattataggcgtgagccaccacgcccagcctatctttttttttttttttaagaaaaggtaatacattgagcagttttcctcccaacctagtccctgtccaatctgtttcctgtcttaaatttattttttattataatttttttttttttttttttttgtgagacagacttttgctcttgttagccaggctggagtgcagtggtgctatcttgactcacggcaacctccacctcccaggttcaagcgattctcctgcctcagcctctggagtagcttggattacaagcgcctgccaccacgcccaactaatttagtgtatttttagtagagacggggtttcttttgttgttgttgttgtttttgagacggagtctcactctattgcccaggctggagtgcaacggcacgatctcggctcactgcaacatctgcctcccgggttcaagcgattctactgcctcagcctcctgagtagctgggattacaggcacacaccaccacacctggctaatttttgtatttttagtagagatggggtttcaccatattggccaggctgatctcgaactcctgacctcgtgatctgcccgccttgacctcccaaagtgataggattacagccatgagccgccctgcccaactaatacctgatattaattttattgtatctcacccagcatggtggctcacacctgtaactccttggcagatcgcttgagcctaagagttcaagaccagcttgggcaacatggtgaaaccccgtctctactaaaaatataaaaatgagctgggcatggtggcgtgcacccgtaatcccagctaactcaggaggctgaggcaggaagaatggcttgaacccgggaggcagaggctgcagtgagctgagatcatgccactgcattccagcctgaaacagagcaagactgtctcaaaagaagtttaagtccagcctcaaactcctggactcaagtgatcctcctgcctcagcctcctgagtagtgcaccggccccatctcaagtgttcagtattcacatgtggcttgttactcccgtattaaagagcaaaaatagagaacgtttctttttcttttttttctttttttttttttttttgagatgggagtttcgctcttgtcgcccaggctggagtgcaatggcacgatcttggctcaccgcaacctctgcctcccaggttcaagcgattctcctgcctcagcctcccaagtagctgggattacagacatgcaccaccatgcctagcaaattttgtttttgtttttgtttttttcttttgagacagacttttgcttttattgccaaggctggagtgcaatggtgcaatctcgactcactgcaacctccgcctcccgggttcaagcaattctcctgcctcagctaattttgtagttttagtagaaacggtttctccatgttagctaggctggtctcaaactcccaacctcaggtgatccgcccgcctcgacctcctaaagtgctggaattacaggcgtgagccactgtgcctggccctaattttgtatttttagtagagatggggtttctccctgttggtcaggctggtctcaaactcccaacctaaggtgatccaccagcctcagcctcccaaactgctgggattataggcatgagccaccgcgcccggggaaatatagggaacatttctatcatggccacgttctcttggacagcaatgccctagcacatagtaagcgcttgctgggaatggagttgttttgggcataaatctgtggcccgccccaagctaagctactttccctctcctctctcagcggagccttaaacaacgcagtgagacagacatctatcatcccattttacaggggaggatactgaggcacacagaggggagtcaccagccagaggatgcatagcctggacacagaggaagttggctagaggccggtcccttccttgggcccctctcattccctccccagaatggaggcaacgccagaatccagcaccggccccatttacccaactctgtacaaagcccttgtccccatgaaattgtatataaatcatccttttctaccagctctggccaggtctgtctatggatgggtgtgaatggggtagttttgatttcagaatcttggtttttgaattgcaatgcaatgtttcattcaatacgtgttttctgaggtcccctggagccccgggccggcgctgggcaatgtcagagcctggatttgagtcagaaccagcccaggtgccccaggtctccttggctatggacgagaagacaagggattcagcgagcctgggtgcaggatgccataggctactatggagataaaacagaggcagtgtgaggggcaactcacccaggcagggagagcagagaggctttccagagccaaatggcattgcgatggtgacaagccagatttggagggggtggggagggcatcctgggcaagggacccagctgaagcaaaggccgacagtccagaaagtgagaagttcctctgaggaacgctcagtggattggtgcagccaggcaactagaagccattccttgatccactcttagagcccctggggaagaccacctccatctgttttacagatggataaactgaggcccagataaggccaagggtttctccaggtcacagtgaagtcgaccagacacaggaatccaaatctctaagcctagaatgggcaaggctccggggctcacgccggtaatcccagcactttgggaggccgaaacgggaggatcgcttgagcccaggagttcgagatcagcctggccaacatgtcagactccccccgttcccgggccacaacaaaatatatatatatatatatattagccaggcatggtggcgcgcccctgtagtccaagttactcgagaggctgaggcaggag